(19) United States
(12) Patent Application Publication
(10) Pub. No.: US 2022/0389109 A1
(43) Pub. Date: Dec. 8, 2022

(54) Management of conditions other than multiple sclerosis in ofatumumab-treated patients

(71) Applicant: Novartis AG, Basel (CH)

(72) Inventors: Martin Merschhemke, Schopfheim (DE); Ratnakar Pingili, Southlake, TX (US)

(21) Appl. No.: 17/753,635

(22) PCT Filed: Sep. 10, 2020
(86) PCT No.: PCT/EP2020/075331

§ 371 Date: Mar. 9, 2022

(30) Foreign Application Priority Data
Sep. 11, 2019 (EP) 19196789.2
Feb. 21, 2020 (EP) 20158885.2
Mar. 16, 2020 (EP) 20163398.9
Apr. 9, 2020 (EP) 20169007.0
May 22, 2020 (EP) 20176051.9

(51) Int. Cl. C07K 16/28; A61K 31/167; A61P 25/28

(52) U.S. Cl. CPC C07K 16/2887; A61K 31/167; A61P 25/28; C07K 2317/21

(57) ABSTRACT

The disclosure relates to methods of providing a multiple sclerosis (MS) treatment that allows controlling conditions other than MS such as infections.

### FIELD

The present invention relates to ofatumumab for treating or preventing (or for use in the treatment or prevention of) relapsing multiple sclerosis (RMS), wherein ofatumumab is used in a patient with a history of previous or ongoing conditions other than multiple sclerosis.

### BACKGROUND OF THE INVENTION

Invading viruses, bacteria or fungi adapt various mechanisms to resist inactivation or destruction by the host. They can sometimes produce toxins to impair the body's defense system or change their shape or outer structural proteins to disguise from being recognized by the immune system (change of antigenicity). Some bacteria may produce adhesion factors that allow them to stick to the mucus membrane and hinder their destruction. Because of these mechanisms, patients with a history of conditions such as previous or ongoing infections may not tolerate or may be particularly sensitive towards immunosuppressive therapies. However, some diseases such as multiple sclerosis (MS) may require immunosuppressive therapies. Thus, there is a need to provide an MS therapy for patients with a history of conditions such as previous or ongoing infections.

These mechanisms to resist inactivation or destruction by the host are more likely to succeed in immunocompromised or immunosuppressed patients (e.g. patients undergoing MS therapy). Nevertheless, it is often not an option to stop immunosuppression because it is part of therapy (e.g. MS therapy). Thus, there is a need to prevent, reduce or alleviate viral, bacterial or fungal infections while allowing immunosuppression to proceed (e.g. in order to treat MS).

Importantly, immunosuppression is usually not compatible with some prophylactic or therapeutic measures such as vaccinations. In some cases, this creates a dilemma for immunocompromised or immunosuppressed patients (e.g. patients undergoing MS therapy). For example, lower urinary tract infections are common adverse events in immunocompromised or immunosuppressed patients (e.g. patients undergoing MS therapy). Viruses are increasingly recognized as the cause of lower urinary tract infection, especially hemorrhagic cystitis, among immunocompromised or immunosuppressed patients. BK virus, adenovirus and cytomegalovirus are predominant pathogens involved in hemorrhagic cystitis. Cidofovir is becoming a drug of choice in viral urinary tract infections because it is active against the most common viral pathogens. However, it may not be desirable to expose patients (e.g. patients undergoing MS therapy) to a high burden of drugs (including cidofovir) if these patients are already undergoing a treatment and/or are immuno-compromised/immunosuppressed such as MS patients undergoing MS therapy. Cidofovir, for example, is known to cause adverse events like nephrotoxicity and neutropenia. Neutropenia may be dangerous for MS patients treated with e.g. fingolimod because it aggravates leukopenia (lymphopenia) caused by fingolimod, thus compromising the immune system. Nephrotoxicity may be dangerous for MS patients treated with e.g. fingolimod because it impairs the body's ability to excrete fingolimod and its metabolites, which may result in a fluid imbalance and associated diseases. Therefore, there has been a need to address adverse events differently. In particular, there has been a need for MS therapies that allow for vaccinations during therapy.

The Epstein-Barr virus (EBV) is one of the most successful pathogens in humans with more than 90% of the adult population persistently infected (Cesarman 2014, Cohen 2015). EBV infection in immunodeficient individuals can be associated with all sorts of diseases, including malignancies, including carcinoma (gastric or nasopharyngeal) or lymphoma (e.g. Hodgkin lymphoma). In an immunocompetent individual, an anti-viral T cell response controls the infection, but EBV remains latent in memory B cells and some other cell types. But success of MS therapy depends on immunosuppressive drugs to prevent inflammation events in the central nervous system (CNS). Thus, in MS patients, current immunosuppression therapies dampen the anti-EBV T cell response, leaving EBV-induced B cell proliferation uncontrolled. Similarly, most individuals are infected with the varicella zoster virus (VZV) as children which causes an episode of chickenpox. The immune system eventually eliminates the virus from most locations, but it remains dormant (or latent) in the ganglia adjacent to the spinal cord (called the dorsal root ganglion) or the trigeminal ganglion in the base of the skull. The disease (shingles) results from virus particles in a single sensory ganglion switching from their latent lysogenic cycles to their active lytic cycles. VZV infections have been reported as an adverse event in MS patients treated with fingolimod (Cohen et al., New England Journal of Medicine 2010; 362: 402-15). Another virus, JC virus, is responsible for the feared complication of Progressive Multifocal Leukoencephalopathy (PML). As for EBV and VZV, JC virus infected cells are under anti-viral T-cell control. Current immunosuppressive agents affecting T-cell function, increase the risk for activation of latent viruses like EB and JC. Thus, there is an urgent need for novel therapies that modulate, prevent or inhibit virus infections, like EBV or JCV infections and associated diseases in MS patients who are particularly vulnerable.

### SUMMARY OF THE INVENTION

A method of treating multiple sclerosis (MS) in patients with a history of previous or ongoing conditions other than MS is provided. It has surprisingly been found that a patient who has a history of previous or ongoing conditions other than multiple sclerosis and who requires treatment or prevention of relapsing multiple sclerosis can be successfully treated with ofatumumab without substantially compromising the management of the condition other than MS. Further, patients can be vaccinated during ofatumumab therapy. This has been completely unexpected because ofatumumab is an anti-CD20 antibody and depletes lymphocytes. Thus, ofatumumab had been expected to negatively affect the immune system, such that previous or ongoing conditions such as previous or ongoing infections cannot be successfully treated in patients on ofatumumab therapy. In line with this expectation, Pescovitz et al. demonstrated that rituximab, another anti-CD20 antibody, leads to a depression of IgM levels, see “B-lymphocyte depletion with rituximab and β-cell function: two-year results”, Diabetes Care, 2014 February; 37(2); 453-9.

### SHORT DESCRIPTION OF THE FIGURES

FIG. 1 describes the set-up of the clinical trial according to example 1 and the measurements of IgG and IgM.

FIG. 2 illustrates the change of serum IgG levels from baseline.

FIG. 3 illustrates the change of serum IgM levels from baseline.

FIG. 4 shows the decrease in IgG levels following administration of an anti-CD20 antibody of the prior art (Ocrelizumab). FIG. 4 was first published as part of T. Derfuss et al.: “Serum immunoglobulin levels and risk of serious infections in the pivotal Phase III trials of ocrelizumab in multiple sclerosis and their open-label extensions”, ECTRIMS Online Library. Derfuss T. Sep. 11, 2019; 279399; 65.

FIG. 5 shows that, after two years (96 weeks), ocrevus treatment (pooled OPERA, see FIG. 4) has led to a reduction of IgG levels by approx. 5% whereas ofatumumab has led to an increase of about 3%.

FIG. 6 shows the treatment groups of study in which a single dose of an anti-CD20 antibody was administered to mice via two different routes (i.v. or s.c.) to investigate the effect of B-cell depletion on the antibody-mediated immunity.

FIG. 7 shows the B-cell depletion and vaccination study design of FIG. 6 in more detail.

FIGS. 8 and 9 show the total B-cell population at Day 14 in a one-dose vaccination study (FIG. 8: B cell gating CD19+, CD3−, CD11b−, LygG/C−; FIG. 9: Proportion and total number of B cells).

FIGS. 10 and 11 show B-cell subtypes at Day 14 in a one-dose vaccination study (FIG. 10: B cell gating; marginal zone CD23−CD21+; Follicular: CD23+IgD+; Germinal center: PNA+IgD−; FIG. 11: Proportion and number of undepleted B cells).

FIG. 12 shows B-cell depletion at day 29 in a two-dose vaccination study, using spleen homogenates.

FIG. 13 shows B-cell depletion at day 29 in a two-dose vaccination study, highlighting the proportion, number and subtypes of B cells.

FIGS. 14 and 15 show pneumococcal-specific immunoglobulin levels (IgG/IgM) in a two-dose vaccination study at Days 16 and 29.

### GENERAL DEFINITIONS

The terms “treatment” or “treat” can be defined as the application or administration of e.g. ofatumumab to a patient, where the purpose is to abolish, reduce or alleviate the symptoms of a disease such as multiple sclerosis (MS). In particular, the term “treatment” comprises the achievement of a clinically meaningful effect for the patient, for example the achievement of a clinical meaningful reduction of the annual relapse rate when treating RMS.

As used herein, a patient can be “in need of” a treatment if such a patient would benefit medically or in terms of the quality of life from such treatment. The term “patient” as used herein can be a mammal, e.g. a primate, preferably a higher primate, especially preferred a human (e.g. a patient having a risk or at risk of having a disorder described herein). Preferably, the patient is an adult.

As used herein, the term “administering” or “administration” of ofatumumab can mean providing ofatumumab to a patient in need of treatment. Administration “in combination with” one or more further therapeutic agents includes simultaneous (concurrent) and consecutive administration in any order and in any route of administration.

As used herein, a “therapeutically effective amount” can refer to an amount of ofatumumab that is effective, i.e. achieves a clinically meaningful effect.

The term “adverse event” (AE) can relate to any untoward medical occurrence in a patient or clinical investigation wherein the subject is administered a pharmaceutical product which does not necessarily have a causal relationship with this treatment.

An adverse event (AE) can therefore be any unfavourable and unintended sign (including an abnormal laboratory finding), symptom or disease temporally associated with the use of a medicinal (investigational) product, whether or not related to the medicinal (investigational) product.

The phrase “therapeutic regimen” can mean the regimen used to treat an illness or to prevent a disease condition or the development of a disease, e.g. the dosing used. A therapeutic regimen may include an induction regimen, a loading regimen and a maintenance regimen.

The phrase “loading regimen” or “loading dose” can refer to a treatment regimen (or the portion of a treatment regimen) that is used for the initial treatment of a disease. In some embodiments, the disclosed methods, uses, kits, processes and regimens employ a loading regimen. In some cases, the loading period is the period until maximum efficacy is reached. The general goal of a loading regimen can be to provide a high level of drug to a patient during the initial period of a treatment regimen. An induction regimen may employ (in part or in whole) a “loading regimen” or “loading dosing”, which may include administering a greater dose of the drug than a physician would employ during maintenance regimen, administering a drug more frequently than a physician would administer the drug during a maintenance regimen or both. Dose escalation may occur during or after an induction regimen.

The phrase “maintenance regimen” or “maintenance dose” can refer to a treatment regimen (or the portion of a treatment regimen) that is used for the maintenance of a patient during treatment of an illness, e.g. to keep the patient in remission for long periods of time (months or years) following the induction period. In some embodiments, the disclosed methods, uses and regimens employ a maintenance regimen. A maintenance regimen may employ continuous therapy (e.g. administering a drug at a regular intervals, e.g. weekly, monthly [every 4 weeks], yearly etc.) or intermittent therapy (e.g. interrupted treatment, intermittent treatment, treatment at relapse or treatment upon achievement of a particular predetermined criteria [e.g. pain, disease manifestation etc.]). Dose escalation may occur during a maintenance regimen.

The term “Multiple Sclerosis Impact Scale (MSIS-29)” is defined as follows: The MSIS-29 version 2 is a 29-item, self-administered questionnaire that includes 2 domains: physical and psychological. Responses were captured on a 4-point ordinal scale ranging from 1 (not at all) to 4 (extremely), with higher scores reflecting greater impact on day-to-day life. The MSIS-29 takes about 5 minutes to complete and the questions are designed to determine the patient's views about the impact of MS on their day-to-day life during the past 2 weeks. Reference is made to Hobart J and Cano S (2009), “Improving the evaluation of therapeutic interventions in multiple sclerosis: the role of new psychometric methods”, Health Technol Assess; 13(12):iii, ix-x, 1-177. NS RO to Hobart J, Lamping D, Fitzpatrick R, et al (2001), “The Multiple Sclerosis Impact Scale (MSIS-29): a new patient-based outcome measure”, Brain; 124(Pt 5):962-73.

The expression “a patient with a history of previous conditions other than multiple sclerosis” can mean that the patient has or had a pre-existing condition. A pre-existing condition is defined as a “medical condition that occurred before a program of health benefits went into effect” (“Billing terminology”. Pittsburgh: University of Pittsburgh Medical Center (UPMC). 2010. Archived from the original on Oct. 3, 2010. Retrieved Jan. 16, 2010). In the context of the present invention, the “program of health benefits” can relate to a therapy comprising the administration of ofatumumab. Thus, the pre-existing condition occurred or started before the commencement of ofatumumab therapy. In a preferred embodiment, ofatumumab therapy is for treating (R)MS.

Vaccination can be the administration of a vaccine to help the immune system develop protection from a disease. Vaccines preferably contain a microorganism or virus in a weakened, live or killed state, or proteins or toxins from the organism. In stimulating the body's adaptive immunity, they help prevent sickness from an infectious disease. When a sufficiently large percentage of a population has been vaccinated, herd immunity results. Vaccination can be the most effective method of preventing infectious diseases. However, if a person is already under treatment to cure, modify a first disease (such as MS) or alleviate the symptoms of a first disease (such as MS), it may be—according to findings in the art—difficult or impossible to vaccinate that person against a second disease.

In a preferred embodiment, the vaccine is not Tetanus toxoid, 13-valent pneumococcal conjugate vaccine (13-PCV), 23-valent pneumococcal polysaccharide vaccine (23-PPV), seasonal quadrivalent influenza vaccine, HPV vaccine and/or hepatitis B vaccine.

RRMS

Relapsing-remitting multiple sclerosis (MS) is characterized by relapses, e.g. defined as a new neurologic deficit or episode of neurologic worsening lasting longer than 24 h in the absence of fever or infection.

There is no apparent progression of the disease during the periods of remission. At different points in time, RRMS can be further characterized as either active (with relapses and/or evidence of new MRI activity) or not active, as well as worsening (a confirmed increase in disability over a specified period of time following a relapse) or not worsening. Reference is made to Lublin 2014, Neurology. 2014 Jul. 15; 83(3): 278-286.

RMS

The term RMS (relapsing multiple sclerosis) encompasses RRMS, SPMS and clinically isolated syndrome (CIS).

Primary Progressive MS (PPMS)

PPMS is characterized by worsening neurologic function (accumulation of disability) from the onset of symptoms, without early relapses or remissions. PPMS can be further characterized at different points in time as either active (with an occasional relapse and/or evidence of new MRI activity) or not active, as well as with progression (evidence of disease worsening on an objective measure of change over time, with or without relapse or new MRI activity) or without progression. References is made to Lublin 2014.

Each person's experience with PPMS will be unique. PPMS can have brief periods when the disease is stable, with or without a relapse or new MRI activity, as well as periods when increasing disability occurs with or without new relapses or lesions on MM.

Secondary Progressive MS (SPMS)

SPMS follows an initial relapsing-remitting course. Most people who are diagnosed with RRMS will eventually transition to a secondary progressive course in which there is a progressive worsening of neurologic function (accumulation of disability) over time. SPMS can be further characterized at different points in time as either active (with relapses and/or evidence of new MM activity) or not active, as well as with progression (evidence of disease worsening on an objective measure of change over time, with or without relapses) or without progression. Reference is made to Lublin 2014.

Each person's experience with SPMS will be unique. SPMS follows after relapsing-remitting MS. Disability gradually increases over time, with or without evidence of disease activity (relapses or changes on MM). In SPMS, occasional relapses may occur, as well as periods of stability.

Clinically Isolated Syndrome (CIS):

Clinically isolated syndrome (CIS) may refer to a single clinical attack of central nervous system (CNS) inflammatory demyelinating symptoms that are suggestive of multiple sclerosis (MS). CIS presentations can be monofocal or multifocal and typically may involve the optic nerve, brainstem, cerebellum, spinal cord or cerebral hemispheres. Reference is made to Miller et al, Clinically isolated syndromes, Lancet Neurol. 2012; 11:157-169.

T1 and T2 lesions

T1 and T2 relate to different MRI methods used to generate magnetic resonance images. Specifically, T1 and T2 refers to the time taken between magnetic pulses and recording of an image. These different methods are used to detect different structures or chemicals in the central nervous system. T1 and T2 lesions refers to whether the lesions were detected using either the T1 or T2 method. A T1 MRI image supplies information about current disease activity by highlighting areas of active inflammation. A T2 MRI image provides information about disease burden or lesion load (the total amount of lesion area, both old and new).

Relapse

Relapses can be defined as a new neurologic deficit or episode of neurologic worsening, preferably lasting longer than 24 h. In other words, relapses can be regarded as discrete episodes (in the art also referred to as “attacks,” “flare-ups” or “exacerbations”) of neurologic dysfunction, preferably lasting at least 24 h. Usually, relapses are followed by full or partial recovery and a period in which there is no symptom progression or accumulation of disability (remission).

Ofatumumab:

Ofatumumab is a human monoclonal antibody for the CD20 protein. Ofatumumab may bind specifically to both the small and large extracellular loops of the CD20 molecule. The Fab domain of ofatumumab may bind to the CD20 molecule and the Fc domain mediates immune effector functions to result in B-cell lysis in vitro. In particular, ofatumumab is a recombinant human monoclonal immunoglobulin G1 (IgG1) antibody that binds to human CD20 expressed on e.g. B cells. Ofatumumab is produced in a murine NS0 cell line and consists of two IgG1 heavy chains and two kappa light chains with a molecular weight of approximately 146 kDa.

Ofatumumab is described in EP 1 558 648 B1 and EP 3 284 753 B1. Further reference is made to the description in the drugbank.ca, accession number DB06650 and to WHO Drug Information, Vol. 20, No. 1, 2006. In an embodiment, the protein chemical formula is C₆₄₈₀H₁₀₀₂₂N₁₇₄₂O₂₀₂₀S₄₄ and the protein average weight is about 146100 Da.

The metabolic pathway of ofatumumab can be degradation to small peptides and amino acids by ubiquitous proteolytic enzymes. Ofatumumab might be eliminated in two ways: a target-independent route as with other IgG molecules and a target-mediated route that is related to binding to B cells.

The half-life of ofatumumab at steady state can be approximately 16 days, in particular following subcutaneous administration of repeated 20 mg doses.

Ofatumumab preferably does not share a common clearance pathway with chemical drugs that are metabolized by the cytochrome P450 system or other drug metabolizing enzymes. Preferably, ofatumumab is not involved in the regulation of the expression of drug metabolizing enzymes.

### DETAILED DESCRIPTION OF THE INVENTION

Prolonged, low levels of immunoglobulin G (IgG) and/or IgM are associated with a heightened risk of infections. This has raised concerns because several MS therapies were found to reduce immunoglobulin levels. Particularly, treatment with B-cell-depleting therapies has been reported to result in decreased serum levels of immunoglobulin (Ig)G, IgM and/or IgA. Since, moreover, MS therapy usually is a life-long therapy, MS patients are at an increased risk of infections.

For example, Ocrelizumab (tradename Ocrevus), an anti-CD20 antibody, may result in higher infection rates in patients receiving it. Ocrevus was approved in the US for the treatment of relapsing MS (RMS) and primary progressive MS (PPMS). Rituximab, another antibody which is similar to ocrelizumab, has been reported to be associated with higher risk of infection, particularly in patients with low levels of IgM or IgG.

In June 2019, the European Commission updated the ocrelizumab prescribing information describing the association between the decrease in immunoglobulins and serious infections as follows: “Treatment with Ocrevus resulted in a decrease in total immunoglobulins over the controlled period of the studies mainly driven by reduction in IgM. Clinical trial data have shown an association between decreased levels of IgG (and less so for IgM or IgA) and serious infections.”

K. Smoot et al. (“The Impact of Ocrelizumab on Immunoglobulin Levels and the Risk of Infection”, Sep. 12, 2019; 278212; P1010) studied ocrelizumab-treated MS patients and reported that there was no evidence of a significant difference in IgM levels between patients who did and did not have infections. However, they emphasized that infections were more commonly seen in patients with lower IgG levels.

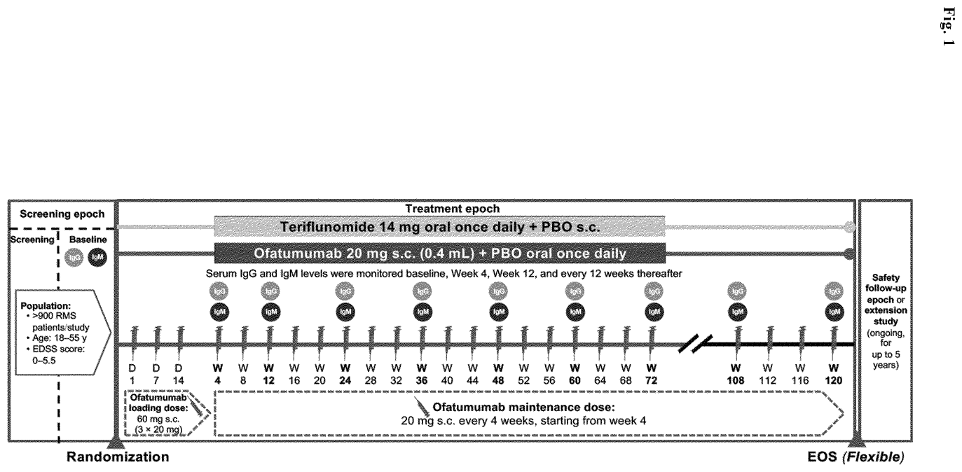

T. Derfuss et al. (“Serum immunoglobulin levels and risk of serious infections in the pivotal Phase III trials of ocrelizumab in multiple sclerosis and their open-label extensions”, ECTRIMS Online Library. Derfuss T. Sep. 11, 2019; 279399; 65) assessed serum Ig levels over 5.5 years. They observed a reduction in serum Ig levels, with an apparent association with increased rates of serious infections. The association was strongest for IgG and less so for IgM or IgA. The reduction in serum Ig levels proceeded at an approximate mean rate of 3-4% per year (see FIG. 1). There was an apparent association between decreased levels of IgG and serious infections.

Taken together, one of the most common adverse events associated with B-cell-depleting therapies such as ocrelizumab therapy in clinical trials was reported to be a reduction of immunoglobulins (e.g. IgM) in the blood.

Therefore, it is completely surprising that ofatumumab therapy is advantageous compared to other B-cell-depleting therapies because it causes reduction of immunoglobulins (e.g. IgM) to a lesser extent. Consequently, the risk of serious infection is unexpectedly lowered in ofatumumab-treated patients. Moreover, the absence or amelioration of negative effects on the immune system opens up new avenues for ofatumumab-treated patients. For example, they may be vaccinated while on ofatumumab therapy. Moreover, they can be treated with ofatumumab despite previous or ongoing conditions other than multiple sclerosis. This is an important clinical benefit which is explained in detail hereinafter.

By contrast, other immunomodulatory or immunosuppressive treatments (such as ocrelizumab) put patients at a higher risk of stronger immune reactions (e.g. due to an activation of the complement system and/or decreased interaction between B cells and T cells and/or altered cytokine production and/or bystander activation). Moreover, these other treatments require more careful timing of vaccination, if possible or recommendable at all (see Tobias Zrzavy et al.: Vaccination in Multiple Sclerosis: Friend or Foe?, FRONTIERS IN IMMUNOLOGY, vol. 10, 1 Jan. 2019, page 1883).

An aspect of the invention relates to ofatumumab for use in the treatment or prevention of relapsing multiple sclerosis (RMS), wherein ofatumumab is used in a patient with a history of previous or ongoing conditions other than multiple sclerosis. The expression “a patient with a history of previous conditions other than multiple sclerosis” means that the patient has or had a pre-existing condition. A pre-existing condition is defined as a “medical condition that occurred before a program of health benefits went into effect” (“Billing terminology”. Pittsburgh: University of Pittsburgh Medical Center (UPMC). 2010. Archived from the original on Oct. 3, 2010. Retrieved Jan. 16, 2010). In the context of the present invention, the “program of health benefits” relates to a therapy comprising the administration of ofatumumab. Thus, the pre-existing condition occurred or started before the commencement of ofatumumab therapy.

The condition other than multiple sclerosis may be one or several of

Nasopharyngitis

Headache

Injection-site reaction

Upper respiratory tract infection

Urinary tract infection

Back pain

Fatigue

Influenza

Nausea

Decreased blood immunoglobulin M or G

Alopecia

Arthralgia

Diarrhoea

Pain in extremity

Depression

Macular edema

Chickenpox (varicella)

head colds

increased gamma-glutamyl transfer

abdominal pain

skin cancer

bradycardia

hemorrhaging focal encephalitis

herpes infection

progressive multifocal leukoencephalopathy (PML)

Hypertension

Paraesthesia.

These conditions other than multiple sclerosis may result from viral, bacterial or other infections as described below.

Alternatively, these conditions other than multiple sclerosis may result from medication such as immunosuppressive drugs. Immunosuppressive drugs include corticosteroids such as prednisone (Deltasone, Orasone), budesonide (Entocort EC), prednisolone (Millipred); Janus kinase inhibitors such as tofacitinib (Xeljanz); calcineurin inhibitors such as cyclosporine (Neoral, Sandimmune, SangCya) and tacrolimus (Astagraf XL, Envarsus XR, Prograf); mTOR inhibitors such as sirolimus (Rapamune) and everolimus (Afinitor, Zortress); IMDH inhibitors such as azathioprine (Azasan, Imuran), leflunomide (Arava) and mycophenolate (CellCept, Myfortic); lymphocyte sequestrants such as fingolimod (Gilenya); biologics such as abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio); monoclonal antibodies such as basiliximab (Simulect), daclizumab (Zinbryta), ocrelizumab (Ocrevus). In a preferred embodiment, the immunosuppressive drug is fingolimod (Gilenya), ocrelizumab (Ocrevus), natalizumab (Tysabri) or rituximab (Rituxan). Fingolimod (Gilenya) is particularly preferred.

As a further alternative, said conditions other than multiple sclerosis may result from diseases such as autoimmune diseases other than multiple sclerosis.

In a preferred embodiment of this aspect of the invention, the history of previous or ongoing conditions other than multiple sclerosis is a history of previous or ongoing infections. The expression “history of previous or ongoing infections” means that the patient has or had a pre-existing infection. A pre-existing infection is defined in analogy to a pre-existing condition as indicated above. In the context of the present invention, the “program of health benefits” relates to a therapy comprising the administration of ofatumumab. Thus, the pre-existing infection occurred or started before the commencement of ofatumumab therapy.

Patients suffering from infections are susceptible to more serious signs and symptoms if they undergo an immunosuppressive therapy. Since anti-CD20 antibodies suppress the immune system, it has been completely surprising that ofatumumab being an anti-CD20 antibody does not trigger or favour more serious signs and symptoms.

The previous or ongoing infection may have been caused by a virus or microorganism selected from the group consisting of

a respiratory syncytial virus (RSV),

an influenza or parainfluenza virus,

a human polyomavirus (BK virus),

an adenovirus,

a rhinovirus,

a corona virus, in particular SARS-CoV-2,

a human herpes virus like a herpes simplex virus (HSV),

a varicella zoster virus (VZV),

an Epstein-Barr virus (EBV),

a cytomegalovirus (CMV),

a betapolyomavirus like John Cunningham virus (JCV),

Bordetella pertussis,

Bordetella parapertussis,

Corynebacterium diphtheriae,

E. coli,

Staphylococcus spec. (e.g. Staphylococcus saprophyticus or Staphylococcus aureus),

Chlamydia trachomatis,

Haemophilus influenzae,

Meningococcus spec.,

Klebsiella spec.,

Pseudomonas spec.,

Enterococcus spec.,

Streptococcus spec.,

yeast (e.g. Candida albicans),

Pneumocystis spec. (e.g. Pneumocystis murina),

Cryptococcus spec. (e.g. Cryptococcus neoformans),

Aspergillus spec.,

Mycoplasma genitalium.

Thus, the history of previous conditions other than multiple sclerosis may be triggered by a viral infection wherein the virus causing infection is, for example, a respiratory syncytial virus (RSV), an influenza or parainfluenza virus, a human polyomavirus (BK virus), an adenovirus, rhinovirus, corona virus, a human herpesvirus like a Herpes simplex virus (HSV), a varicella zoster virus (VZV), a EBV or Cytomegalovirus (CMV) or a betapolyomavirus like John Cunningham virus (JCV).

Three of the four types of influenza viruses affect humans: Type A, Type B and Type C. Type D has not been known to infect humans but is believed to have the potential to do so. Usually, the virus is spread through the air from coughs or sneezes. It can also be spread by touching surfaces contaminated by the virus and then touching the eyes, nose or mouth. A person may be infectious to others both before and during the time they are showing symptoms. Yearly vaccinations against influenza are recommended by the World Health Organization (WHO) for those at high risk and by the Centers for Disease Control and Prevention (CDC) for those six months of age and older. However, patients who are susceptible to undergo or commencing an immunosuppressive therapy are usually not eligible or responsive to vaccinations because their immune system is suppressed. Since anti-CD20 antibodies suppress the immune system, it was completely surprising that ofatumumab being an anti-CD20 antibody does not impair vaccinations against the influenza virus.

Antiviral drugs such as the neuraminidase inhibitor oseltamivir, among others, have been used to treat influenza. It has surprisingly been found that said drugs can be used in patients who are treated with or commence treatment with ofatumumab. Thus, an aspect of this invention is based on the surprising finding that ofatumumab does not impair influenza control, in contrast to other anti-CD20 therapies.

Coronaviruses are a group of viruses that cause diseases in mammals and birds. In humans, coronaviruses cause respiratory tract infections that are typically mild, such as some cases of the common cold (among other possible causes, predominantly rhinoviruses), though rarer forms can be lethal, such as SARS, MERS and COVID-19. There are yet to be vaccines or antiviral drugs to prevent or treat human coronavirus infections. It is envisaged that patients who are infected with corona virus, e.g. SARS-CoV-2, can be treated with or commence treatment with ofatumumab.

There are two types of herpes simplex virus, type 1 (HSV-1) and type 2 (HSV-2). HSV-1 more commonly causes infections around the mouth while HSV-2 more commonly causes genital infections. They are transmitted by direct contact with body fluids or lesions of an infected individual. After infection, the viruses are transported along sensory nerves to the nerve cell bodies, where they reside lifelong. Causes of recurrence may include decreased immune function, stress and sunlight exposure. Thus, patients are susceptible to recurrence if they undergo an immunosuppressive therapy. Since anti-CD20 antibodies suppress the immune system, it was completely surprising that ofatumumab being an anti-CD20 antibody does not trigger or favour recurrence.

Daily antiviral medication taken by someone who has the infection can also reduce spread. There is no available vaccine and once infected there is no cure. Paracetamol (acetaminophen) and topical lidocaine may be used to help with the symptoms. Treatments with antiviral medication such as acyclovir or valaciclovir can lessen the severity of symptomatic episodes. It has surprisingly been found that said drugs can be used in patients who are treated with or commence treatment with ofatumumab. Thus, an aspect of this invention is based on the surprising finding that ofatumumab does not impair HSV control in contrast to other anti-CD20 therapies.

Human alpha herpesvirus 3 (HHV-3), usually referred to as the varicella-zoster virus (VZV), is one of nine herpesviruses known to infect humans. It causes chickenpox (varicella), a disease most commonly affecting children, teens and young adults, and shingles (herpes zoster) in adults; shingles is rare in children. VZV multiplies in the lungs and causes a wide variety of symptoms. After the primary infection (chickenpox), the virus goes dormant in the nerves, including the cranial nerve ganglia, dorsal root ganglia and autonomic ganglia. Many years after the person has recovered from chickenpox, VZV can reactivate to cause neurologic conditions. Within the human body it can be treated by a number of drugs and therapeutic agents including acyclovir for the chicken pox, famciclovir, valaciclovir for the shingles, zoster-immune globulin (ZIG) and vidarabine. VZV immune globulin is also a treatment. Acyclovir is frequently used as the drug of choice in primary VZV infections and beginning its administration early can significantly shorten the duration of any symptoms. It has surprisingly been found that said drugs and therapeutic agents can be used as a VZV therapy in patients who are treated with or commence treatment with ofatumumab. The patient may even be vaccinated against VZV during ofatumumab therapy. Thus, an aspect of this invention is based on the surprising finding that ofatumumab does not impair VZV control, in contrast to other anti-CD20 therapies. Therefore, the disclosure relates to a method of preventing a VZV-associated disease in a patient, comprising administering to said patient a therapeutically effective dose of ofatumumab.

In an embodiment of the invention, ofatumumab is not administered to patients having an active HBV infection.

Epstein—Barr virus (EBV) is a human herpesvirus 4 (HHV4) and belongs to the genus Lymphocryptovirus within the subfamily of gamma herpes viruses. These viruses establish latent infections of their host cells and induce proliferation of the latently infected cells (reviewed in Roizman B. Herpesviridae: general description, taxonomy and classification. In: Roizman B, editor. The herpesviruses. London: Plenum Press, 1996:1_/23.). EBV is associated with a still growing spectrum of clinical disorders, ranging from acute and chronic inflammatory diseases to lymphoid and epithelial malignancies. Epstein—Barr virus is associated with lymphoproliferative diseases, a type of diseases in which different types of lymphoid cells like T-cells, B-cells or natural killer (NK) cells are infected with the Epstein Barr virus. The infected cells divide excessively and develop various lymphoproliferative disorders (LPD, non-cancerous, pre-cancerous and cancerous). These LPDs include infectious mononucleosis and subsequent disorders that may occur thereafter. Non-LPD but EBV-associated diseases include malignancies, sarcomas, multiple sclerosis, systemic lupus erythematosus, Hodgkin and non-Hodgkin lymphomas, nasopharyngeal carcinoma, gastric carcinoma, leiomyo-sarcoma and the “Alice in Wonderland syndrome” (Middeldorp et al., Critical Reviews in Oncology/Hematology 45 (2003) 1-/36 2003).

An aspect of this invention is based on the surprising finding that ofatumumab does not impair EBV control in vitro, in contrast to other anti-CD20 therapies. Hence, the surprising finding that an immunosuppressive anti-CD20 therapy does not impair EBV control is the precondition and basis for the development of therapeutic methods in patients being at risk of developing EBV-associated diseases.

Cytomegalovirus (CMV) is a genus of viruses in the order Herpesvirales, in the family Herpesviridae, in the subfamily Betaherpesvirinae. Humans and monkeys serve as natural hosts. Human betaherpesvirus 5 (HCMV, human cytomegalovirus, HHV-5), which is the species that infects humans. Diseases associated with HHV-5 include mononucleosis and pneumonia. Most people infected with CMV who are otherwise healthy experience few if any signs and symptoms. However, a few babies with congenital CMV who appear healthy at birth can develop signs over time—sometimes not for months or years after birth. The most common of these late-occurring signs are hearing loss and developmental delay. A small number of babies may also develop vision problems. Moreover, people with a weakened immunity might experience more serious signs and symptoms affecting:

Eyes,

Lungs,

Liver,

Esophagus,

Stomach,

Intestines,

Brain.

In a preferred embodiment, controlling viral infection (viral control) is possible, i.e. proceeds without substantial or at least acceptable delays or limitations, despite (the commencement of) ofatumumab therapy. The term viral control or controlling viral infection also refers to the above described treatment, wherein the viral load in whole blood of a treated patient is below 5000 copies of the viral genome/μg DNA, below 4500 copies/μg DNA, below 4000 copies/μg DNA, below 3500 copies/μg DNA, below 3000 copies/μg DNA, below 2500 copies/μg DNA, below 2000 copies/μg DNA, below 1500 copies/μg DNA or below 1000 copies/μg DNA. In a preferred embodiment the above-described viral load in whole blood is maintained for at least a period of 6 months, for at least a period of 9 months, for at least a period of 12 months, for at least a period of 15 months, for at least a period of 18 months, for at least a period of 21 months, for at least a period of 24 months, for at least a period of 3 years, for at least a period of 4 years, for at least a period of 5 years, for at least a period of 6 years, for at least a period of 7 years, for at least a period of 8 years or longer after the transplantation has taken place.

The term viral control or controlling viral infection also refers to the above-described treatment, wherein the viral load in plasma is below 3000 copies/100 μl, below 2500 copies/100 μl, below 2000 copies/100 μl, below 1500 copies/100 μl or below 1000 copies/100 μl. In a preferred embodiment the above described viral load in plasma is maintained for at least a period of 6 months, for at least a period of 9 months, for at least a period of 12 months, for at least a period of 15 months, for at least a period of 18 months, for at least a period of 21 months, for at least a period of 24 months, for at least a period of 3 years, for at least a period of 4 years, for at least a period of 5 years, for at least a period of 6 years, for at least a period of 7 years, for at least a period of 8 years or longer after the transplantation has taken place.

Furthermore, the term viral control or controlling viral infection as used herein also refers to the treatment of a patient, in particular wherein the patient is an MS patient, more particular a patient in need of immuno-suppression, with a therapeutically effective dose of ofatumumab, wherein a reduced viral titer or viral load or viral infection status is obtained, wherein the viral load (e.g. viral DNA load) is reduced by at least by 20%, by at least by 30%, by at least by 40%, by at least by 50%, by at least by 60%, by at least by 70%, by at least by 80%, by at least by 90% or by more than 90%. In a preferred embodiment, the reduced viral load is maintained for at least a period of 6 months, for at least a period of 9 months, for at least a period of 12 months, for at least a period of 15 months, for at least a period of 18 months, for at least a period of 21 months, for at least a period of 24 months, for at least a period of 3 years, for at least a period of 4 years, for at least a period of 5 years, for at least a period of 6 years, for at least a period of 7 years, for at least a period of 8 years or longer after the commencement of ofatumumab therapy.

In a preferred embodiment, the treatment according to the invention allows for prevention of a virus-associated disease by providing suitable measures (e.g. medication or vaccination) that are usually not used during an immunosuppressive treatment. Since anti-CD20 antibodies suppress the immune system, it has been completely surprising that ofatumumab being an anti-CD20 antibody does not impair the prevention of a virus-associated disease. The terms “prevent” or “preventing” generally refer to prophylactic or preventative treatment; they are concerned with delaying the onset of or preventing the onset of the disease, disorders and/or symptoms associated thereto. The term “preventing a virus-associated diseases” as used herein refers to the outcome of the treatment of a patient, in particular wherein the patient is an MS patient, more particular a patient in need of immuno-suppression, with a therapeutically effective dose of ofatumumab, wherein the patient does not develop a virus-associated disease, in particular the patient does not develop lymphoproliferative disorders (LPD, non-cancerous, pre-cancerous and cancerous, including infectious mononucleosis and subsequent disorders that may occur thereafter, or non-LPD but virus-associated diseases including malignancies, sarcomas, multiple sclerosis, systemic lupus erythematosus and the “Alice in Wonderland syndrome”. The term “preventing virus-associated diseases” also refers to the outcome of the treatment of a patient as described above, wherein the patient does not develop a virus-associated disease as described herein for at least a period of 12 months, for at least a period of 18 months, for at least a period of 24 months, for at least a period of 3 years, for at least a period of 4 years, for at least a period of 5 years, for at least a period of 6 years, for at least a period of 7 years, for at least a period of 8 years or longer after the commencement of ofatumumab therapy.

As mentioned before, the history of previous conditions other than multiple sclerosis may be triggered by a bacterial infection wherein the bacterium causing infection is, for example, Bordetella pertussis, Bordetella parapertussis, Corynebacterium diphtheriae, E. coli, Staphylococcus spec. (e.g. Staphylococcus saprophyticus or Staphylococcus aureus), Chlamydia trachomatis, Haemophilus influenzae, Meningococcus spec., Klebsiella spec., Pseudomonas spec., Enterococcus spec., Streptococcus spec.

Bordetella pertussis is a Gram-negative coccobacillus and the causative agent of pertussis or whooping cough. Its virulence factors include pertussis toxin, adenylate cyclase toxin, filamentous hæmagglutinin, pertactin, fimbria, and tracheal cytotoxin.

Diphtheria is an infection caused by the bacterium Corynebacterium diphtheriae. Complications may include myocarditis, inflammation of nerves, kidney problems, and bleeding problems due to low levels of platelets. Myocarditis may result in an abnormal heart rate and inflammation of the nerves may result in paralysis.

Escherichia coli, also known as E. coli, is a Gram-negative bacterium that is commonly found in the lower intestine. Most E. coli strains are harmless, but some serotypes can cause serious food poisoning in their hosts and are occasionally responsible for food contamination incidents. Virulent E. coli strains can cause gastroenteritis, urinary tract infections, neonatal meningitis, hemorrhagic colitis, and Crohn's disease. Common signs and symptoms include severe abdominal cramps, diarrhea, hemorrhagic colitis, vomiting and sometimes fever. In rarer cases, virulent strains are also responsible for bowel necrosis (tissue death) and perforation without progressing to hemolytic-uremic syndrome, peritonitis, mastitis, sepsis and Gram-negative pneumonia.

Some strains of E. coli, for example O157:H7, can produce Shiga toxin (classified as a bioterrorism agent). The Shiga toxin causes inflammatory responses in target cells of the gut, leaving behind lesions which result in the bloody diarrhea that is a symptom of a Shiga toxin-producing E. coli (STEC) infection. This toxin further causes premature destruction of the red blood cells, which then clog the body's filtering system, the kidneys, in some rare cases (usually in children and the elderly) causing hemolytic-uremic syndrome (HUS), which may lead to kidney failure and even death.

Uropathogenic E. coli (UPEC) is one of the main causes of urinary tract infections. It is part of the normal microbiota in the gut and can be introduced in many ways.

Enterotoxigenic E. coli (ETEC) is the most common cause of traveler's diarrhea, with as many as 840 million cases worldwide in developing countries each year. The bacteria, typically transmitted through contaminated food or drinking water, adheres to the intestinal lining, where it secretes either of two types of enterotoxins, leading to watery diarrhea.

Certain strains of E. coli are a major cause of foodborne illness. Enterohemorrhagic E. coli (EHEC) bacteria lead to hemolytic-uremic syndrome (HUS), a medical emergency that requires urgent treatment.

Staphylococcus is a genus of Gram-positive bacteria in the family Staphylococcaceae in the order Bacillales. Staphylococcus can cause a wide variety of diseases in humans and animals through either toxin production or penetration. Staphylococcal toxins are a common cause of food poisoning, for they can be produced by bacteria growing in improperly stored food items. The most common sialadenitis is caused by staphylococci, as bacterial infections.

A chlamydia infection is a sexually transmitted infection caused by the bacterium Chlamydia trachomatis. Most people who are infected have no symptoms. When symptoms do develop this can take a few weeks following infection to occur. The infection can spread to the upper genital tract in women, causing pelvic inflammatory disease, which may result in future infertility or ectopic pregnancy. Repeated infections of the eyes that go without treatment can result in trachoma, a common cause of blindness in the developing world. Chlamydia can be spread during vaginal, anal or oral sex and can be passed from an infected mother to her baby during childbirth.

In a preferred embodiment, controlling bacterial infection (bacterial control) is possible, i.e. proceeds without substantial or at least acceptable delays or limitations, despite (the commencement of) ofatumumab therapy. The term bacterial control or controlling bacterial infection also refers to the above described treatment, wherein bacteremia is prevented or reduced. Bacteremia is most commonly diagnosed by blood culture, in which a sample of blood drawn from the vein by needle puncture is allowed to incubate with a medium that promotes bacterial growth. If bacteria are present in the bloodstream at the time the sample is obtained, the bacteria will multiply and can thereby be detected.

In another embodiment, the history of previous conditions other than multiple sclerosis may be triggered by a fungal infection wherein the fungus causing infection is yeast (e.g. Candida albicans), Pneumocystis spec. (e.g. Pneumocystis murina), Cryptococcus spec. (e.g. Cryptococcus neoformans) and Aspergillus spec.

Candida albicans is an opportunistic pathogenic yeast that is a common member of the human gut flora. It is usually a commensal organism but it can become pathogenic in immunocompromised individuals under a variety of conditions. It is one of the few species of the genus Candida that causes the human infection candidiasis, which results from an overgrowth of the fungus. Candidiasis is for example often observed in HIV-infected patients. C. albicans is the most common fungal species isolated from biofilms either formed on (permanent) implanted medical devices or on human tissue. C. albicans, C. tropicalis, C. parapsilosis and C. glabrata are together responsible for 50-90% of all cases of candidiasis in humans. A mortality rate of 40% has been reported for patients with systemic candidiasis due to C. albicans. Recent studies indicate that C. albicans can cross the blood brain barrier.

The genus Pneumocystis represents related fungal species that are members of the phylum/division Ascomycota, the subphylum Taphrinomycotina, class Pneumocystidomycetes, order Pneumocystidales and family Pneumocystidaceae, all within the kingdom of fungi. Clusters of cases of Pneumocystis pneumonia (PCP) that have been reported in immunocompromised patients. Pneumocystis infection has global distribution among humans and most individuals show serologic evidence of infection by 2 years of age. The incidence of PCP is related to the extent of immunosuppression, especially impairment in cell-mediated immunity, as evidenced by the frequent occurrence of PCP in patients with AIDS.

In a preferred embodiment, controlling fungal infection (fungal control) is possible, i.e. proceeds without substantial or at least acceptable delays or limitations, despite (the commencement of) ofatumumab therapy.

In another embodiment, the history of previous conditions other than multiple sclerosis may be triggered by a mycoplasmatic infection wherein the mycoplasm causing infection is Mycoplasma genitalium.

Mycoplasma is a genus of bacteria that lack a cell wall around their cell membranes. This characteristic makes them naturally resistant to antibiotics that target cell wall synthesis (like the beta-lactam antibiotics). Mycoplasma genitalium is a sexually transmitted, small and pathogenic bacterium that lives on the skin cells of the urinary and genital tracts in humans. It can cause negative health effects in men and women.

Furthermore, different organisms require varying times of onset from when they enter the body to when symptoms occur (incubation time). Some of the common pathogens for upper respiratory infection and their respective incubation times are the following:
Rhinoviruses, 1-5 days;
Group A streptococci, 1-5 days;
Influenza and parainfluenza viruses, 1-4 days;
Respiratory syncytial virus (RSV), 7 days;
Whooping cough (pertussis), 7-21 days;
Diphtheria, 1-10 days; and
Epstein-Barr virus (EBV), 4-6 weeks.

Thus, some of these pathogens allow intervention for up to several weeks after entering the body before they cause disease. Therefore, the treatment strategy may be changed during the incubation time.

Thus, according to another aspect of the disclosure, a method of preventing, reducing or alleviating an adverse event in a patient at risk of developing such an adverse event (e.g. MS patients) is provided, comprising administering to said patient ofatumumab, wherein the patient is a multiple sclerosis (MS) patient who has been treated with a disease-modifying therapy other than ofatumumab. In one embodiment, ofatumumab treatment is commenced when the patient was exposed to a risk of viral, bacterial or fungal infection. Alternatively, ofatumumab treatment is commenced when signs of a viral, bacterial or fungal infection (e.g. DNA) are detected but the patient does not yet show any symptoms. The adverse event may be one or several of injection-related reactions, nasopharyngitis, headache, injection-site reaction, upper respiratory tract infection, urinary tract infection, back pain, fatigue, influenza, nausea, decrease in blood immunoglobulin M or G, alopecia, arthralgia, diarrhoea, pain in extremity, depression, macular edema, chickenpox (varicella), head colds, increased gamma-glutamyl transfer, abdominal pain, skin cancer, bradycardia, hemorrhaging focal encephalitis, herpes infection, progressive multifocal leukoencephalopathy (PML), hypertension, paraesthesia.

In another embodiment, the method according to the disclosure is a method wherein the patient at risk of developing an infection-associated disease is immunosuppressed or receiving immunomodulatory drugs. In one embodiment the method according to the disclosure is a method wherein the adverse event is cancer or a lymphoproliferative disease.

In a preferred embodiment of this aspect of the invention, the ofatumumab-treated patient is vaccinated during ofatumumab therapy. In another embodiment of the invention, the ofatumumab-treated patient is vaccinated during ofatumumab therapy, said patient being without a history of previous or ongoing conditions other than multiple sclerosis.

The vaccination may be against any one of

a thinovirus,

a respiratory syncytial virus (RSV),

an influenza or parainfluenza virus,

a human polyomavirus (BK virus),

an adenovirus,

a human herpesvirus like a Herpes simplex virus (HSV),

a varicella zoster virus (VZV),

an Epstein-Barr virus (EBV),

a Cytomegalovirus (CMV),

a betapolyomavirus like John Cunningham virus (JCV),

Bordetella pertussis,

Bordetella parapertussis,

Corynebacterium diphtheriae,

E. coli,

Staphylococcus saprophyticus,

Staphylococcus aureus,

Chlamydia trachomatis,

Klebsiella spec.,

Pseudomonas spec.,

Enterococcus spec.,

Streptococcus spec.,

yeast (e.g. Candida albicans),

Pneumocystis spec. (e.g. Pneumocystis murina),

Cryptococcus spec. (e.g. Cryptococcus neoformans),

Aspergillus spec.,

Mycoplasma genitalium,

a corona virus, in particular SARS-CoV-2.

It has been completely unexpected that vaccinations can be successful in ofatumumab-treated patients because ofatumumab is an anti-CD20 antibody and depletes lymphocytes. Thus, ofatumumab had been expected to negatively affect the immune system, such immunoglobulins, which are necessary to acquire immunity after vaccination. In line with this expectation, Pescovitz et al. demonstrated that rituximab, another anti-CD20 antibody, leads to a depression of IgM levels, see “Pescovitz et al., “B-lymphocyte depletion with rituximab and β-cell function: two-year results”, Diabetes Care, 2014 February; 37(2); 453-9.

Hence, surprisingly vaccination can be carried out during ofatumumab therapy. Vaccination can be carried out in patients with or without a history of previous or ongoing conditions other than multiple sclerosis. Hence, a further subject of the present invention is ofatumumab for use in the treatment or prevention of relapsing multiple sclerosis, wherein a vaccination is performed during ofatumumab therapy. Generally, embodiments (e.g. the amount and dosage regimens of Ofatumumab administration) described for vaccinating patients with a history can also apply for patients without a history.

In another embodiment of this aspect of the invention, the patient with a history of previous or ongoing conditions other than multiple sclerosis may have a history of transient ischemic attack (TIA);

history of cerebral infarction without residual deficits;

history of thrombotic stroke without lasting effects;

history of thrombotic stroke without residual deficits;

history of transient ischemic attack;

history of ischemic stroke without residual deficits;

history of nonatherosclerotic stroke without residual deficits;

history of parietal cerebrovascular accident;

history of cerebrovascular accident without residual deficits;

history of embolic stroke without deficits;

history of embolic stroke without lasting effects;

history of embolic transient ischemic attack;

history of haemorrhagic cerebrovascular accident without residual deficits;

history of atherosclerotic cerebrovascular accident without residual deficits;

history of cardioembolic stroke.

Here, the expression “a patient with a history of . . . ” means that the patient has or had a pre-existing condition (e.g. TIA). A pre-existing condition is defined as a “medical condition that occurred before a program of health benefits went into effect” (“Billing terminology”. Pittsburgh: University of Pittsburgh Medical Center (UPMC). 2010. Archived from the original on Oct. 3, 2010. Retrieved Jan. 16, 2010). In the context of the present invention, the “program of health benefits” relates to a therapy comprising the administration of ofatumumab. Thus, the pre-existing condition (e.g. TIA) occurred or started before the commencement of ofatumumab therapy.

It is envisaged that the patient with a history of previous or ongoing conditions other than multiple sclerosis followed a treatment to cure, alleviate or abolish said condition before commencing ofatumumab therapy. This treatment may comprise administration of glucocorticoids, such as cortisol, and/or nonsteroidal anti-inflammatory drugs (NSAIDs), such as ibuprofen (Motrin, Advil) and naproxen (Naprosyn) or COX-2 inhibitors, such as celecoxib and/or immune-suppressing drugs.

In a preferred embodiment of this aspect of the invention, the previous or ongoing condition is an autoimmune disease other than multiple sclerosis. The autoimmune disease other than multiple sclerosis may be selected from

Type 1 diabetes,

Rheumatoid arthritis (RA),

Psoriasis/psoriatic arthritis,

Systemic lupus erythematosus (SLE),

Inflammatory bowel disease,

Addison's disease,

Graves' disease,

Sjögren's syndrome,

Hashimoto's thyroiditis,

Myasthenia gravis,

Autoimmune vasculitis,

Pernicious anemia,

Celiac disease.

Thus, the autoimmune disease may be rheumatoid arthritis (RA). In this case, the rheumatoid arthritis (RA) may be treated with a disease-modifying antirheumatic drug (DMARD) selected from the group of methotrexate, hydroxychloroquine, sulfasalazine, leflunomide, TNF-alpha inhibitors (certolizumab, infliximab and etanercept), abatacept, anakinra, rituximab and tocilizumab.

Alternatively, the autoimmune disease may be psoriasis. The psoriasis may be treated with methotrexate, ciclosporin, hydroxycarbamide, fumarates such as dimethyl fumarate, retinoids, anti-TNF therapies such as infliximab, adalimumab, golimumab, and certolizumab pegol, etanercept, ixekizumab, ustekinumab, guselkumab, efalizumab and alefacept.

In a preferred embodiment of this aspect of the invention, the history of previous or ongoing conditions other than multiple sclerosis is a history of hospitalization. The expression “history of hospitalization” means that the patient was or has been in hospital before the commencement of ofatumumab therapy. In particular, it is envisaged that the history of hospitalization includes a history of intensive care and/or surgery. Preferably, the term “history of hospitalization” does not encompass giving birth. The patient with a history of surgery may be treated with immunosuppressive agents other than ofatumumab. Said immunosuppressive agents may be selected from the group consisting of glucocorticoids (such as cortisone, prednisone, dexamethasone and hydrocortisone), cytostatics (such as methotrexate, anthracyclines, mitomycin C, bleomycin, mithramycin), antibodies (such as basiliximab (Simulect) and daclizumab (Zenapax)) or drugs acting on immunophilins (such as tacrolismus and cyclosporine).

Alternatively, the history of previous conditions other than multiple sclerosis may be a history of surgery. The expression “history of surgery” means that the patient underwent or has been undergoing surgical interventions before the commencement of ofatumumab therapy. The patient with a history of surgery may be treated with immunosuppressive agents other than ofatumumab. Said immunosuppressive agents may be selected from the group consisting of glucocorticoids (such as cortisone, prednisone, dexamethasone and hydrocortisone), cytostatics (such as methotrexate, anthracyclines, mitomycin C, bleomycin, mithramycin), antibodies (such as basiliximab (Simulect) and daclizumab (Zenapax)) or drugs acting on immunophilins (such as tacrolismus and cyclosporine).

Ofatumumab may be used in the treatment or prevention of relapsing multiple sclerosis (RMS) in a geriatric patient. The geriatric patient may suffer from age-related macular degeneration (AMD) or from Alzheimer's disease or atherosclerosis or from benign prostatic hyperplasia (BPH).

Ofatumumab may be used in the treatment or prevention of relapsing multiple sclerosis (RMS) in a paediatric patient.

In a preferred embodiment of this aspect of the invention, the condition other than multiple sclerosis is cancer or a lymphoproliferative disease.

In all embodiments described so far, ofatumumab may be administered when serum neurofilament light chain (NfL) concentration is 4 to 13 pg/mL. Neurofilament light chain is a neurofilament protein that in humans is encoded by the NEFL gene. Neurofilament light chain is a biomarker that can be measured with immunoassays in cerebrospinal fluid and plasma and reflects axonal damage in a wide variety of neurological disorders. It has surprisingly become a useful marker for disease monitoring in multiple sclerosis. This has been demonstrated by the ASCLEPIOS I and II studies. 1,882 patients with MS, between the ages of 18 and 55 years, with an Expanded Disability Status Scale (EDSS) score between 0 and 5.5 were enrolled. The studies were conducted in over 350 sites in 37 countries. Additional secondary endpoints included confirmed disability improvement at 6 months, serum levels of neurofilament light chain (NfL), and rate of brain volume loss.

Generally, dosing regimens for ofatumumab are described in WO 2018/033841 and may be applied to the present invention.

The MSIS-29 (see definition above) is a clinically useful and scientifically sound measure of the impact of MS from the patient's perspective suitable for clinical studies and epidemiological studies. It is considered a reliable, valid and responsive PRO (Patient Reported Outcomes) measure that complements other indicators of disease severity used to improve our understanding of the impact of MS.

In the present invention it was unexpectedly found that the administration of ofatumumab leads to an advantageous reduction of the MS impact scale MSIS-29 as defined below.

In this regard a further subject of the present invention is ofatumumab for use in the treatment or prevention of relapsing multiple sclerosis, wherein ofatumumab reduces the MSIS-29 score. Preferably, ofatumumab reduces the MSIS-29 score by at least 1.5, more preferably at least 2.0, still more preferably at least 2.5 within 24 months. The reduction might be up to 3.0 or 3.5 or 4.0.

In all embodiments described so far, ofatumumab may be administered at a dose of 10 to 30 mg every 4 weeks, preferably 20 mg every 4 weeks. Such a dose can be referred to as maintenance dose.

In a preferred embodiment, ofatumumab can be administered irrespective of body weight, sex, age, race or baseline B-cell count. For example, it is preferred that a 35-year-old woman having a body weight of 60 kg receives the same dose as a 50-year old man having a body weight of 90 kg. In particular, body weight, sex, age, race or baseline B-cell count do not have a clinically meaningful effect on the pharmacokinetics of ofatumumab.

In a preferred embodiment, ofatumumab is administered to patients who discontinued earlier DMT, e.g. anti-CD20 therapy, because of side effects such as severe infusion-related reactions or recurrent infections.

Ofatumumab may be administered by injection. In a preferred embodiment it is administered subcutaneously. It was surprisingly found that subcutaneous injection is advantageous compared to other parenteral forms of administration, e.g. compared to intravenous injection.

In a preferred embodiment ofatumumab can be administered with a loading dose. A loading dose can be regarded as an initial higher dose of a drug that may be given at the beginning of a course of treatment before dropping down to a lower maintenance dose. In a preferred embodiment 20 mg ofatumumab at days 1, 7 and 14 may be administered as a loading dose. In a particularly preferred embodiment 20 mg ofatumumab at weeks 0, week 1 and week 2 may be administered as a loading dose.

In a preferred embodiment of the present invention the loading dose is 10-30 mg, preferably 20 mg ofatumumab.

The preferred dosage of ofatumumab is:
initial dosing of 20 mg by subcutaneous injection at Weeks 0, 1 and 2, followed by
subsequent dosing of 20 mg by subcutaneous injection once monthly starting at Week 4.

If an injection of ofatumumab is missed, it should preferably be administered as soon as possible without waiting until the next scheduled dose. Subsequent doses should be administered at the recommended intervals.

Alternatively, ofatumumab is administered without a loading dose.

In a preferred embodiment of the present invention, ofatumumab is administered to a patient who has been treated with a disease-modifying therapy other than ofatumumab.

In a particularly preferred embodiment, the disease-modifying therapy other than ofatumumab is dimethyl fumarate (DMF). Preferably, DMF is administered in a daily dose of 120 mg to 480 mg, in particular 480 mg.

In a particularly preferred embodiment of the present invention the disease-modifying therapy other than ofatumumab is laquinimod. Preferably, laquinimod is administered in a daily dose of 0.2 to 1.0 mg, preferably 0.6 mg.

In a particularly preferred embodiment of the present invention the disease-modifying therapy other than ofatumumab is teriflunomide. Preferably, teriflunomide is administered in a daily dose of 6 to 18 mg, preferably 14 mg.

In a preferred embodiment of the present invention the disease-modifying therapy other than ofatumumab is administered by injection. Examples of suitable DMTs are natalizumab, rituximab, ocrelizumab, alemtuzumab, daclizumab, and glatiramer acetate.

In a preferred embodiment of the present invention, the disease-modifying therapy other than ofatumumab is natalizumab. Preferably, natalizumab is administered by intravenous injections every four weeks at a dose of 100 to 500 mg, preferably, 300 mg.

In a preferred embodiment of the present invention, the disease-modifying therapy other than ofatumumab is daclizumab. Preferably, daclizumab is administered in a dose of 50 to 250 mg, preferably 150 mg s.c. once monthly.

In a preferred embodiment of the present invention, the disease-modifying therapy other than ofatumumab is glatiramer acetate. Preferably, glatiramer acetate is administered in a dose of 20 mg/mL by s.c. injection once-daily regimen, or 40 mg/mL by s.c. injection 3-times-per-week.

In a preferred embodiment of the present invention, the disease-modifying therapy other than ofatumumab is rituximab. Preferably, rituximab is administered in a dose of 500 or 1,000 mg every 6-12 months, in particular intravenously.

In a preferred embodiment of the present invention, the disease-modifying therapy other than ofatumumab is ocrelizumab. Preferably, ocrelizumab is administered in a dose of 600 mg every 6 months, in particular intravenously.

Preferably, patients have been previously treated with at least 2, e.g. 2-5 consecutive courses of intravenous ocrelizumab or rituximab. The last dose may be administered e.g. 4-9 months before ofatumumab is administered.

According to the present invention, there is maintained efficacy of ofatumumab in patients with RMS transitioning from intravenous anti-CD20 therapies.

In a preferred embodiment, ofatumumab is administered to patients with suboptimal response to anti-CD20 therapy in the previous 6 months (e.g. relapse, ≥2 active gadolinium-enhancing [Gd+] lesions, any new/enlarging T2 lesions, clinical worsening) and/or to patients or who discontinued anti-CD20 therapy because of adverse events, e.g. severe infusion-related reactions or recurrent infections.

In a preferred embodiment of the present invention, the disease-modifying therapy other than ofatumumab is alemtuzumab. Preferably, alemtuzumab is administered in a dose of 12 mg/day, administered as intravenous infusion.

In a preferred embodiment of the present invention, ofatumumab is administered at a dose of 10 to 30 mg every 4 weeks, preferably 20 mg every 4 weeks. Preferably, ofatumumab is administered by subcutaneous injection (s.c.).

Ofatumumab can be administered in form of pharmaceutical formulations, e.g. formulations described in WO 2009/009407.

In a preferred embodiment, ofatumumab injection is a sterile, preservative-free solution for subcutaneous use. Preferably, each 20 mg/0.4 mL prefilled pen or prefilled syringe delivers 0.4 mL of solution. Preferably, each 0.4 mL contains 20 mg of ofatumumab and arginine (4 mg), disodium edetate (0.007 mg), polysorbate 80 (0.08 mg), sodium acetate trihydrate (2.722 mg), sodium chloride (1.192 mg) and Water for Injection, USP with a pH of 5.5. Hydrochloric acid may be added to adjust pH.

In a preferred embodiment the ofatumumab formulation is intended for patient self-administration, preferably by subcutaneous injection.

In a preferred embodiment said formulation is administered in the abdomen, thigh or outer upper arm subcutaneously. In a preferred embodiment said formulation is not administered into moles, scars or areas where the skin is tender, bruised, red, hard or not intact.

In an embodiment, the first injection of said ofatumumab formulation may be performed under the guidance of a healthcare professional. If injection-related reactions occur, symptomatic treatment is recommended. Before administration, the pen or prefilled syringe is preferably removed from the refrigerator and allowed to reach room temperature, e.g. for about 15 to 30 minutes. In a preferred embodiment the ofatumumab formulation of the present invention is a clear to slightly opalescent and colorless to slightly brownish-yellow solution available as follows:
Injection: 20 mg/0.4 mL in a single-dose prefilled pen, e.g. Sensoready® pen
Injection: 20 mg/0.4 mL in a single-dose prefilled syringe.

In a preferred embodiment, a subcutaneous ofatumumab dose of 20 mg every 4 weeks leads to a mean AUCₜₐᵤ of about 400 to 550, more preferably 450 to 500, e.g. 483 mcg h/mL and/or to a mean Cₘₐₓ of 1.0 to 2.5, more preferably 1.2 to 1.7, e.g. 1.43 mcg/mL at steady state. In a preferred embodiment, the volume of distribution at steady-state can be 4.5 to 6.5, more preferably 5.0 to 6.0, e.g. 5.42 L following subcutaneous administration of repeated ofatumumab 20 mg doses.

After subcutaneous administration, ofatumumab can be absorbed via the lymphatic system.

In all embodiments described so far, relapsing multiple sclerosis may be selected from relapsing remitting multiple sclerosis (RRMS) and secondary progressive multiple sclerosis (SPMS). Currently, the United States National Multiple Sclerosis Society and the Multiple Sclerosis International Federation, describes four types of MS (revised in 2013):

Clinically isolated syndrome (CIS)

Relapsing-remitting MS (RRMS)

Primary progressive MS (PPMS)

Secondary progressive MS (SPMS)

In a preferred embodiment of the invention, ofatumumab is administered for the treatment of relapsing forms of multiple sclerosis (MS), to include clinically isolated syndrome, relapsing-remitting disease and active secondary progressive disease, preferably in adults.

Relapsing-remitting MS is characterized by unpredictable relapses followed by periods of months to years of relative quiet (remission) with no new signs of disease activity. Deficits that occur during attacks may either resolve or leave problems, the latter in about 40% of attacks and being more common the longer a person has had the disease. This describes the initial course of 80% of individuals with MS. When deficits always resolve between attacks, this is sometimes referred to as benign MS, although people will still build up some degree of disability in the long term. On the other hand, the term malignant multiple sclerosis is used to describe people with MS having reached significant level of disability in a short period. The relapsing-remitting subtype usually begins with a clinically isolated syndrome (CIS). In CIS, a person has an attack suggestive of demyelination, but does not fulfil the criteria for multiple sclerosis. 30 to 70% of persons who experience CIS later develop MS. Thus, CIS patients may be patients as described herein, i.e. patients requiring treatment or prevention of relapsing multiple sclerosis.

Primary progressive MS occurs in approximately 10-20% of individuals, with no remission after the initial symptoms. It is characterized by progression of disability from onset with no or only occasional and minor remissions and improvements. The usual age of onset for the primary progressive subtype is later than of the relapsing-remitting subtype. It is similar to the age that secondary progressive usually begins in relapsing-remitting MS, around 40 years of age.

Secondary progressive MS occurs in around 65% of those with initial relapsing-remitting MS, who eventually have progressive neurologic decline between acute attacks without any definite periods of remission. Occasional relapses and minor remissions may appear. The most common length of time between disease onset and conversion from relapsing-remitting to secondary progressive MS is 19 years.

Atypical variants of MS have been described; these include tumefactive multiple sclerosis, Balo concentric sclerosis, Schilder's diffuse sclerosis, and Marburg multiple sclerosis. There is debate on whether they are MS variants or different diseases. Some diseases previously considered MS variants like Devic's disease are now considered outside the MS spectrum.

In all embodiments described so far, a premedication may be administered to the patient before the first dose of ofatumumab is administered. Said premedication may comprise acetaminophen, antihistamines and/or steroids. It may be administered 30 to 60 minutes prior to ofatumumab injection.

Alternatively, no premedication may be administered prior to the first dose of ofatumumab.

The effect of the prevention of a previous or ongoing condition, e.g. an infection-associated disease, can be assessed by standard routine health checks performed by physicians and other skilled persons using state of the art assays and technologies to diagnose and monitor diseases. The skilled person is aware of respective state of the art diagnosis technologies that can be applied for the above-described purpose. For example, the viral load or infection status can be analysed by e.g. measuring the viral DNA load, wherein the viral DNA quantification can be analysed in whole blood, plasma and/or B-cells. The skilled person is aware of technologies to analyse the viral load and infection status in patients. Viral DNA load can be assessed by analysing expression of viral genes.

The disclosure furthermore relates to a method of reducing the likelihood that a patient will develop an adverse event, comprising administering to the patient a therapeutically effective dose of ofatumumab. The term “reducing the likelihood” as used herein in the context of the development of an adverse event refers to the outcome of the treatment of a patient, in particular wherein the patient is an MS patient, more particular a patient in need of immuno-suppression with a therapeutically effective dose of ofatumumab, wherein the patient has a reduced risk of developing an adverse event. The adverse event may be one or several of injection-related reactions, nasopharyngitis, headache, injection-site reaction, upper respiratory tract infection, urinary tract infection, back pain, fatigue, influenza, nausea, decrease in blood immunoglobulin M or G, alopecia, arthralgia, diarrhoea, pain in extremity, depression, macular edema, chickenpox (varicella), head colds, increased gamma-glutamyl transfer, abdominal pain, skin cancer, bradycardia, hemorrhaging focal encephalitis, herpes infection, progressive multifocal leukoencephalopathy (PML), hypertension, paraesthesia.

### EXAMPLES

### Example 1

Design/Methods

APLIOS was a 12-week, open-label, Phase 2 bioequivalence study. Patients received ofatumumab 20 mg (0.4 mL) s.c. loading doses on Days 1, 7 and 14, and maintenance doses every 4 weeks from Week 4 via a prefilled syringe or an autoinjector pen (SensoReady). Changes in B and T-cell subsets were analysed longitudinally in blood samples of a group of patients using fluorescence-activated cell sorting (FACS).

Results

Ofatumumab treatment showed rapid and sustained depletion in total B cells (CD19+ CD45+) measured on Day 4 and Day 7. The median total B-cell levels decreased to ≤5 cells/μL by Day 7 through Day 14 of the loading regimen and was maintained for the remaining study duration. A more efficient depletion of memory B cells (CD19+ CD45+ CD27+) was observed compared with naïve B cells (CD19+ CD45+ IgD+ CD27− CD38^{dim}). A specific subset of CD20+ CD3+ T cells was also rapidly and potently depleted, consistent with the previously reported findings of a primate study.

Conclusions

Ofatumumab 20 mg s.c. led to a rapid and sustained depletion of both CD20+ B and CD20+ T cells in patients with RMS. The differential impact on specific subsets, i.e. depletion of memory B-cells while sparing naïve B cells, is likely relevant to both the efficacy and long-term safety of ofatumumab in the pathophysiology of MS.

### Example 2

Background

Ofatumumab, the first fully-human anti-CD20 monoclonal antibody, demonstrated superior efficacy versus teriflunomide in Phase 3 ASCLEPIOS I/II trials, see ECTRIMS Online Library, Hauser S. et al. 09/13/19; 279581; 336. MS patients on ofatumumab had a reduction in annualized relapse rate (ARR) by 50.5% (0.11 vs. 0.22) and 58.5% (0.10 vs. 0.25) compared to Aubagio®* (teriflunomide) (both studies p<0.001) in ASCLEPIOS I and II studies respectively. Ofatumumab showed a highly significant suppression of gadolinium (Gd) T1 lesions when compared to Aubagio®, demonstrating a profound suppression of new inflammatory activity. Ofatumumab showed a relative risk reduction of 34.4% in 3-month confirmed disability progression (CDP) (p=0.002) and 32.5% in 6-month CDP (p=0.012) versus Aubagio® in pre-specified pooled analyses.

Objective

To determine serum immunoglobulin (Ig) levels and investigate association between IgG or IgM levels and new therapeutic options, multiple sclerosis patients were treated with ofatumumab.

Methods

ASCLEPIOS I and II are double-blind, double-dummy, active comparator-controlled, parallel-group, innovative, adaptive design, multicentre trials. Patients were randomised (1:1) to receive either ofatumumab 20 mg sc injections every 4 weeks (after an initial loading regimen of 20 mg sc doses on Days 1, 7 and 14) or teriflunomide 14 mg orally once daily, for up to 30 months. The studies have flexible durations, with termination occurring in the blinded core treatment epoch according to pre-specified criteria. Patients aged 18-55 years with an Expanded Disability Status Scale (EDSS) score (according to Kurtzke, Neurology. 1983, November; 33(11): 1444-52) of 0-5.5 at screening who experienced ≥1 relapse in the past year or ≥2 relapses in the past 2 years or a positive gadolinium-enhancing (Gd+) MRI scan during the year before randomisation were included.

Serum IgG/IgM levels were monitored at baseline, Week (W) 4, W12 and every 12 weeks, see FIG. 1. Lower limit of normal (LLN) was defined as IgG, 7 g/L and IgM, 0.4 g/L. Outcomes included proportion of patients with notably low IgG/IgM levels and association between notably low IgG/IgM levels and incidence of infections. Reference is made to FIG. 1.

Results regarding IgG and IgM levels

| | | OMB 20 mg | | | TER 14 mg | | |
|---|---|---|---|---|---|---|---|
| | | N = 946 | | | N = 936 | | |
| Visit- | | Base | Post | Change | Base | Post | Change |
| window | Statistics | Immunoglobulin G (g/L), Plasma/Serum | | | | | |
| Baseline | n | 945 | | | 934 | | |
| | Mean | 10.2 | | | 10.2 | | |
| Week 4 | n | 938 | 938 | 938 | 926 | 926 | 926 |
| | Mean | 10.2 | 9.9 | −0.2 | 10.2 | 9.7 | −0.6 |
| Week 12 | n | 925 | 925 | 925 | 917 | 917 | 917 |
| | Mean | 10.2 | 10.1 | −0.1 | 10.2 | 9.2 | −1.1 |
| Week 24 | n | 902 | 902 | 902 | 887 | 887 | 887 |
| | Mean | 10.2 | 9.9 | −0.4 | 10.2 | 9.0 | −1.3 |
| Week 36 | n | 873 | 873 | 873 | 862 | 862 | 862 |
| | Mean | 10.2 | 9.7 | −0.5 | 10.3 | 8.9 | −1.4 |
| Week 48 | n | 846 | 846 | 846 | 828 | 828 | 828 |
| | Mean | 10.2 | 9.8 | −0.5 | 10.3 | 9.1 | −1.3 |
| Week 60 | n | 817 | 817 | 817 | 802 | 802 | 802 |
| | Mean | 10.2 | 10.0 | −0.2 | 10.3 | 9.3 | −1.0 |
| Week 72 | n | 799 | 799 | 799 | 775 | 775 | 775 |
| | Mean | 10.2 | 10.2 | 0.0 | 10.3 | 9.6 | −0.7 |
| Week 84 | n | 584 | 584 | 584 | 556 | 556 | 556 |
| | Mean | 10.2 | 10.3 | 0.1 | 10.3 | 9.8 | −0.5 |
| Week 96 | n | 370 | 370 | 370 | 363 | 363 | 363 |
| | Mean | 10.2 | 10.5 | 0.3 | 10.3 | 10.0 | −0.3 |
| Week 108 | n | 217 | 217 | 217 | 196 | 196 | 196 |
| | Mean | 10.1 | 10.3 | 0.2 | 10.3 | 10.1 | −0.3 |
| Week 120 | n | 96 | 96 | 96 | 82 | 82 | 82 |
| | Mean | 10.2 | 10.5 | 0.3 | 10.5 | 10.4 | −0.1 |

Base = Baseline; Change = Post Baseline − Baseline.

At each visit-window, only patients with a value at both Baseline and that visit-window are included.

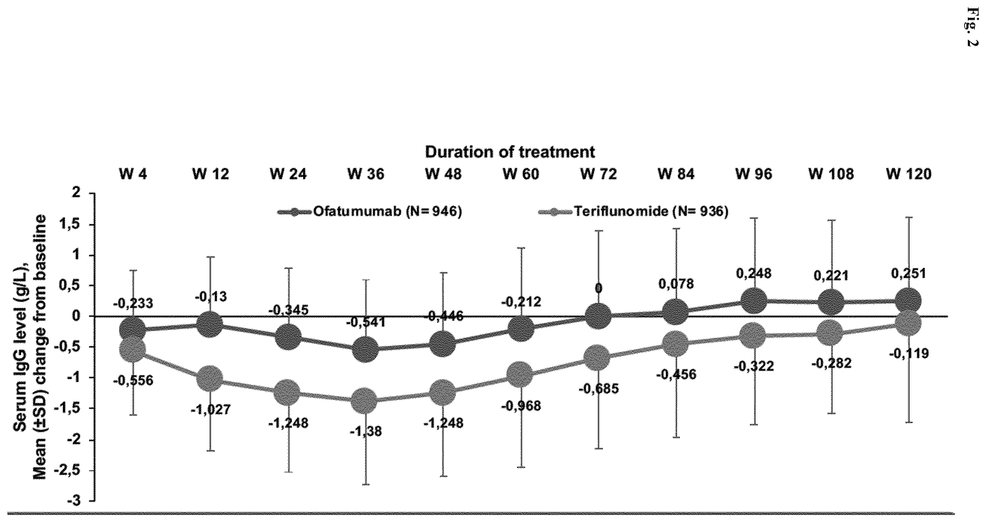

Change in serum IgG levels from baseline is shown in FIG. 2. Change in serum IgM levels from baseline is shown in FIG. 3.

In ofatumumab-treated patients, there is no decrease of IgG levels at week 72 and thereafter. Moreover, there is a turning point at week 36, at which time the trend of decreasing IgG levels reverses, eventually resulting in net increases in IgG starting around week 72.

The IgG decrease is both less pronounced and shorter in ofatumumab-treated patients as compared with teriflunomide-treated patients.

Results Infections: Advantageous low incidence of infections was found.

Conclusion:

Ig levels, in particular IgG levels, were unexpectedly high and incidence of infections was advantageous low.

### Example 3

Methods

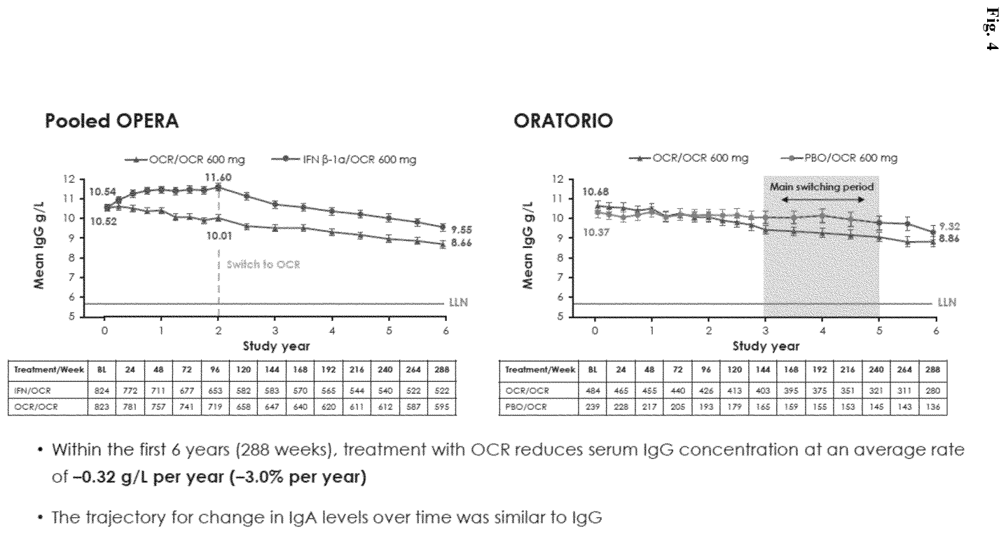

In a metanalysis, the results of Example 3 and the results obtained by Derfuss et al. were compared, see FIG. 5.

Results

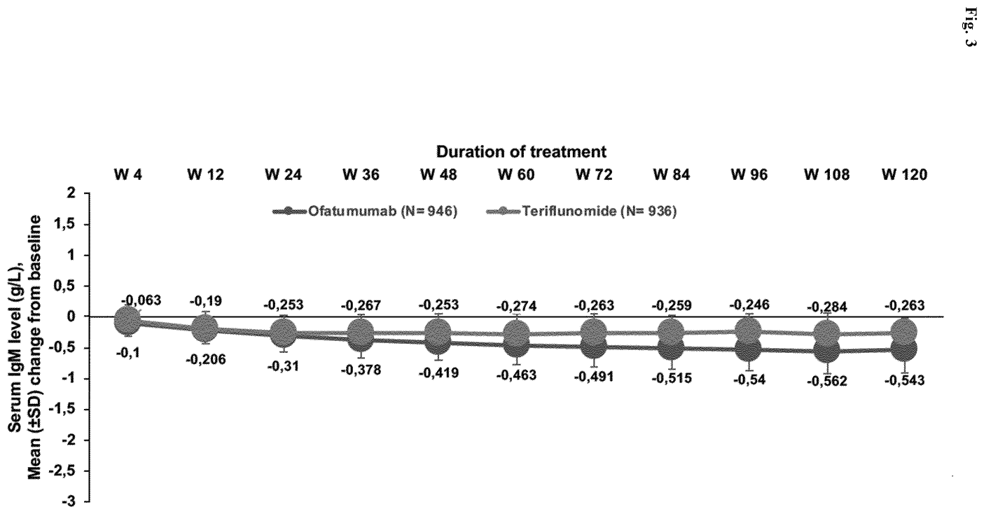

After two years (96 weeks), ocrevus treatment (pooled OPERA, see FIG. 4) has led to a reduction of IgG levels by approx. 5%, whereas ofatumumab has led to an increase of about 3%, see FIG. 2.

Conclusion

Ofatumumab sustains and even increases IgG levels on the long term, whereas ocrelizumab may lead to a rather continuous reduction of IgG levels.

### Example 4

Ofatumumab is administered s.c. to RRMS patients. If injection-related reactions occurs, symptomatic treatment is provided. For patients who experienced pain, redness or itching, hydrocortisone cream is applied after the injection to help with itching, redness and swelling. A pain reliever is also be used.

Observations

The level of (total) IgG in ofatumumab-treated patients with injection-related reactions is expected to be comparable to the level in ofatumumab-treated patients without injection-related reactions and without hydrocortisone treatment. Overall, no evidence of combined immunosuppression or toxicity of ofatumumab and hydrocortisone is expected.

A marked decrease in the follicular B-cell subtype is expected to be observed with ofatumumab treatment, while the marginal zone and germinal center B-cell subtypes are expected to be less affected.

### Example 5

a) Animal Study

A single dose of an anti-CD20 antibody (mIgG1) was administered to mice (C57BL/6 female mice, aged 6 weeks) via two different routes of administration (i.v. or s.c.) to investigate the effect of B-cell depletion on the antibody-mediated immunity to Streptococcus pneumoniae. B cells were depleted by administration of 50 μg/mouse of anti-CD20 (mIgG1, n=8 per group) either via i.v. or s.c. route of administration. Mice without B-cell depletion received the same concentration of isotype control antibody (s.c.). Mice were vaccinated with pneumococcal 13-valent conjugate vaccine Prevnar13° (20 μL/mouse i.p.):
One-dose vaccination study: One dose of Prevnar13°
Two-dose vaccination study: Two doses of Prevnar13°
Control animals (neither depleted nor vaccinated) received PBS (phosphate buffer saline) (i.p.) (see FIG. 6).

Assessment

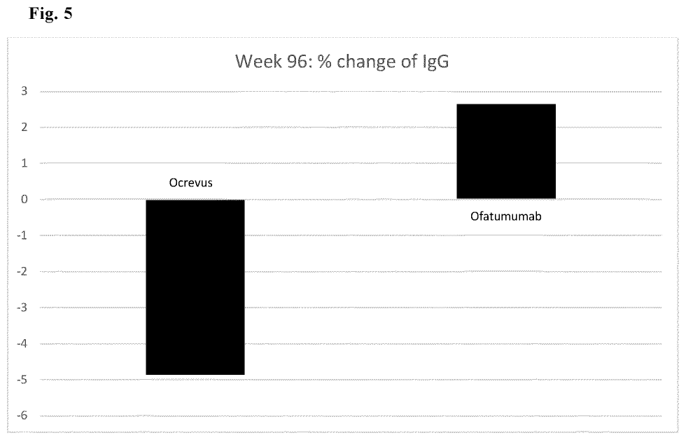

The serum pneumococcal-specific IgG levels were measured at Day 16 (after the first dose of the vaccine) and Day 29 (endpoint) by whole-cell ELISA on pneumococcus (TIGR4 strain)-coated plates (see FIG. 7)

The pneumococcal bacteria were incubated with mice sera (Day 29) and the antibody binding on the pneumococcal surface was measured by flow cytometry (FACS); pneumococcal-specific IgG and IgM levels were measured by a serum deposition assay (see FIG. 7).

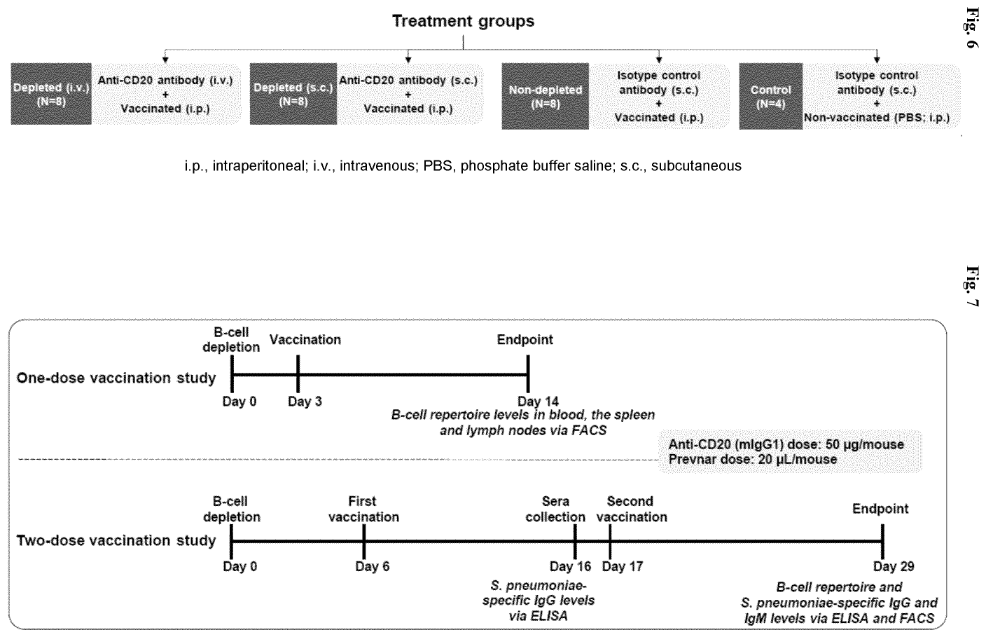

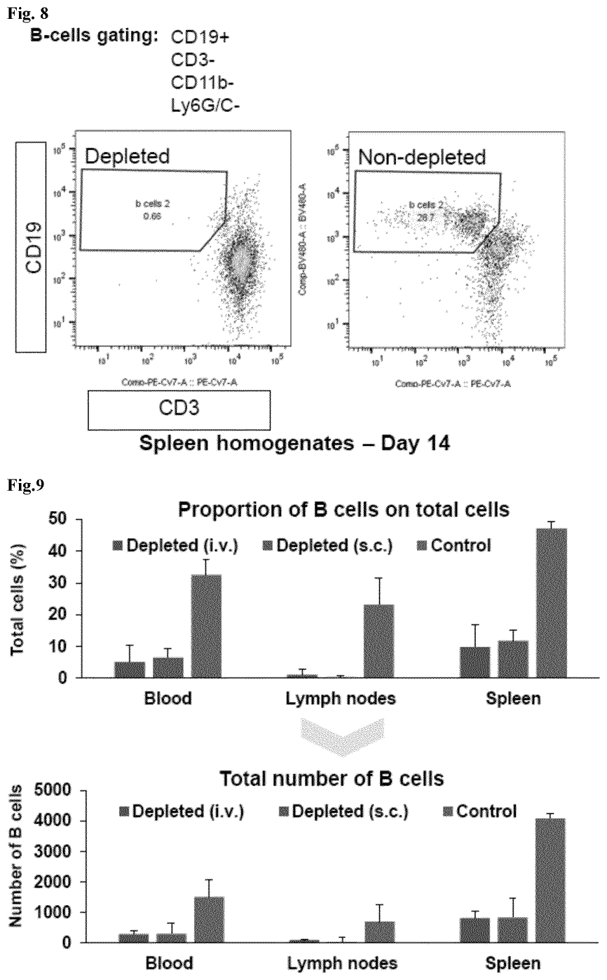

Blood, the spleen and lymph nodes were analyzed to observe the effect on the B-cell repertoire levels at Day 14. After 14 days of anti-CD20 antibody treatment, the number of B cells was still around 20% of the B-cell count in untreated groups (see FIGS. 8 and 9).

One-dose vaccination study: B-cell subtypes at Day 14

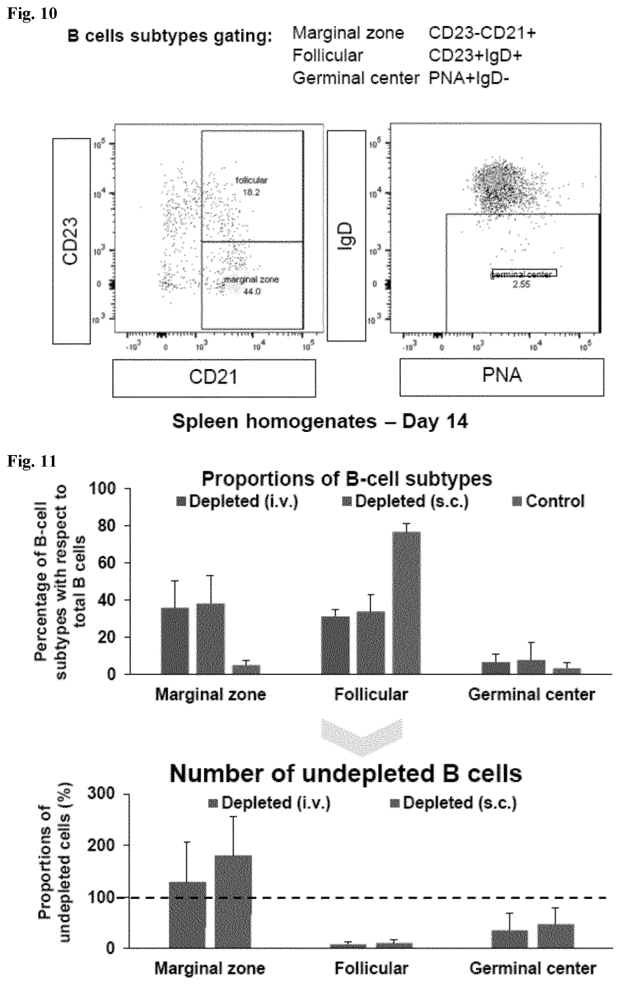

A marked decrease in the follicular B cell-subtypes was observed in both i.v. and s.c. anti-CD20 treatment groups (see FIGS. 10 and 11).

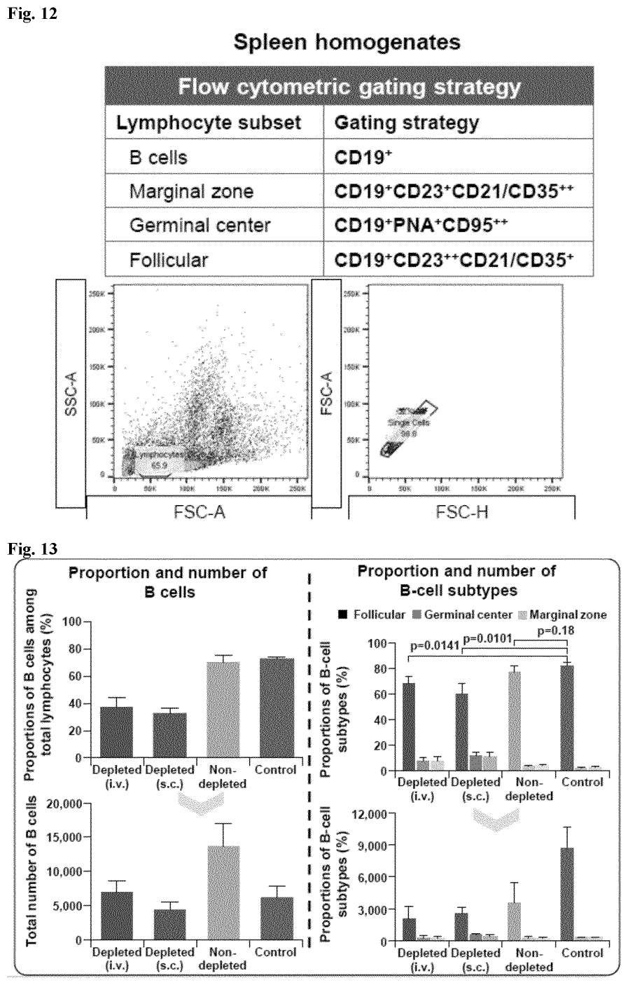

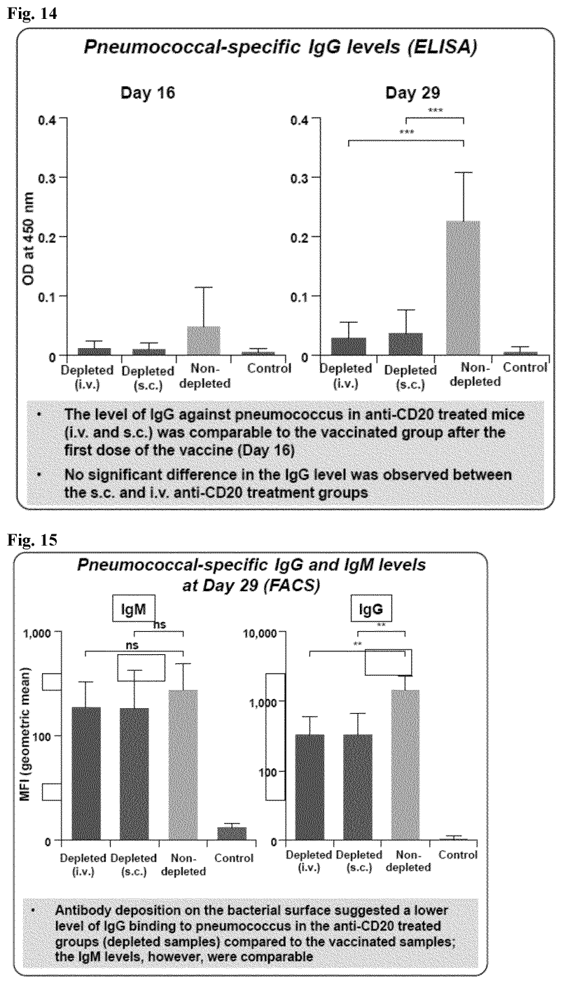

Two-dose vaccination study: B-cell depletion at Day 29
Only 60% of the B-cell population was reconstituted after 4 weeks of anti-CD20 treatment (see FIG. 12).
No significant differences in the B-cell subtypes were observed between the s.c. and i.v. anti-CD20 treatment groups (see FIG. 13).
Pneumococcal-specific immunoglobulin levels (IgG/IgM)
   The level of IgG against pneumococcus in anti-CD20 treated mice (i.v. and s.c.) was comparable to the vaccinated group after the first dose of the vaccine (Day 16, see FIG. 14).
   No significant difference in the IgG level was observed between the s.c. and i.v. anti-CD20 treatment groups (see FIG. 14).
   Antibody deposition on the bacterial surface suggested a lower level of IgG binding to pneumococcus in the anti-CD20 treated groups (depleted samples) compared to the vaccinated samples; the IgM levels, however, were comparable (see FIG. 15).

Conclusions
Route of administration does not influence the undepleted B-cell populations.
A marked decrease in the follicular B-cell subtype was observed with anti-CD20 treatment, while the marginal zone and germinal center B-cell subtypes seemed to be less affected.
The B-cell population was not fully reconstituted after 4 weeks of anti-CD20 treatment.
B-cell depletion reduced the pneumococcal-specific IgG levels, while the reduction in IgM levels was much lower.

b) Clinical Trial

100 patients with relapsing-remitting MS receive ofatumumab s.c.
during a loading dose regimen comprising administering 20 mg ofatumumab at day 0, day 7 and day 14 of the dosage regimen; and
during a maintenance dose regimen comprising administering 20 mg ofatumumab starting at week four of the dosage regimen and continuing thereafter every four weeks for the duration of the treatment protocol.

The general clinical state of the patient is investigated weekly by physical and laboratory examination. Disease state and changes in disease progression are assessed every 2 months by radiological examination (MRI) and physical examination.

For those patients who suffer from infections as an adverse event, blood samples will be taken for laboratory analysis. If the analysis confirms an infection by Staphylococcus aureus, the respective patient will be treated with oral trimethoprim-sulfamethoxazole. Ofatumumab treatment will be continued.

During treatment with trimethoprim-sulfamethoxazole, blood samples are collected every other day. Analyses of these samples is expected to reveal that the level of IgG against Staphylococcus aureus in ofatumumab-treated patients is comparable, as evidenced by a meta-analysis, to those reported in the literature for infected subjects not having MS and not treated with ofatumumab. Moreover, ofatumumab-treated patients are expected to recover more quickly from infection as compared with previous trials.

### Example 6

a) Animal Study

In mice (C57BL/6 female mice, aged 6 weeks), the middle cerebral artery (MCA) was occluded using the endovascular filament model (as described in Hata R, Mies G, Wiessner C, Fritze K, Hesselbarth D, Brinker G, et al. A reproducible model of middle cerebral artery occlusion in mice: hemodynamic, biochemical, and magnetic resonance imaging. J Cereb Blood Flow Metab. 1998; 18:367-375. doi: 10.1097/00004647-199804000-00004). The occlusion is known to cause infarcts in the MCA territory. After reperfusion by withdrawal of the occluding filament, blood flow was promptly restored. A control group was subjected to a mock MCA occlusion.

One week later, a single dose of an anti-CD20 antibody (mIgG1) was administered s.c. to a subset of both groups of mice (MCA occlusion group and mock group). B cells were depleted by administration of 50 μg/mouse of anti-CD20 mIgG1, n=8 per group. Mice without B-cell depletion received the same concentration of isotype control antibody (s.c.).

A marked decrease in the follicular B-cell subtype was observed with anti-CD20 treatment (in both groups), while the marginal zone and germinal center B-cell subtypes seemed to be less affected.

Recovery after MCA occlusion (including tissue repair) seemed to be unaffected by anti-CD20 treatment.

b) Clinical Trial

A first group of patients is monitored after an ischemic stroke. A sub-group of the patients are expected to have difficulty with urinary leakage or are not able to empty their bladder completely because of muscle weakness. For these reasons, a catheter will be placed inside the bladder. However, there is an increased risk of urinary tract infections related to the use of a catheter. These infections will be treated with one of
Trimethoprim/sulfamethoxazole (Bactrim, Septra, others),
Fosfomycin (Monurol),
Nitrofurantoin (Macrodantin, Macrobid),
Cephalexin (Keflex) or
Ceftriaxone.

A second group of patients consists of ofatumumab-treated MS patients. A sub-group of the patients develops adverse events, including urinary tract infections. These infections are treated with one of
Trimethoprim/sulfamethoxazole (Bactrim, Septra, others),
Fosfomycin (Monurol),
Nitrofurantoin (Macrodantin, Macrobid),
Cephalexin (Keflex) or
Ceftriaxone.

Conclusions
A marked decrease in the follicular B-cell subtype is expected to beobserved with ofatumumab treatment, while the marginal zone and germinal center B-cell subtypes are expected to be less affected.
No significant difference is expected with respect to the management of urinary tract infections.
Correlation of data suggests that ofatumumab can be safely administered after a stroke, even after adverse events such as urinary tract infections have occurred.

### Example 7

MS patients are treated with either ofatumumab or Ocrevus (ocrelizumab). Selected patients having psoriatic arthritis are monitored under a special study program. A sub-group of the patients has more severe disease activity and/or does not respond well to NSAIDs. Some of them cannot take a disease-modifying antirheumatic drugs (DMARD) such as
Leflunomide (Arava),
Methotrexate (Otrexup, Rasuvo, Rheumatrex, Trexall) or
Sulfasalazine (Azulfidine).

Thus, they are treated with an immunosuppressant, i.e. one of
Azathioprine (Imuran, Azasan) or
Cyclosporine (Gengraf, Neoral, Sandimmune).

Observations:

The level of IgG in ofatumumab-treated patients is expected to be significantly higher than in Ocrevus-treated patients.

A marked decrease in the follicular B-cell subtype is expected with ofatumumab treatment, while the marginal zone and germinal center B-cell subtypes seemed to be less affected.

After treatment with the immunosuppressant, significantly more adverse events (e.g. infections) is expected in the Ocrevus group.

Comparable management of psoriatic arthritis is expected in both groups.

### REFERENCES

Diabetes Care. 2014 February; 37(2):453-9. doi: 10.2337/dc13-0626. Epub 2013 Sep. 11. B-lymphocyte depletion with rituximab and β-cell function: two-year results. Pescovitz MD1, Greenbaum C J, Bundy B, Becker D J, Gitelman S E, Goland R, Gottlieb P A, Marks J B, Moran A, Raskin P, Rodriguez H, Schatz D A, Wherrett D K, Wilson D M, Krischer J P, Skyler J S; Type 1 Diabetes TrialNet Anti-CD20 Study Group.
Cohen et al., New England Journal of Medicine 2010; 362: 402-15: Oral Fingolimod or Intramuscular Interferon for Relapsing Multiple Sclerosis.
Cesarman E (2014). Gammaherpesviruses and lymphoproliferative disorders. Annu Rev Pathol, 9(349-372).
Cohen J I (2015). Primary Immunodeficiencies Associated with EBV Disease. Curr Top Microbiol Immunol, 390(Pt 1):241-265.

1. Ofatumumab for use in the treatment or prevention of relapsing multiple sclerosis (RMS), wherein ofatumumab is used in a patient with a history of previous or ongoing conditions other than multiple sclerosis.

2. Ofatumumab for use according to claim 1, wherein the condition other than multiple sclerosis is one or several of
Injection-related reaction,
Nasopharyngitis,
Headache,
Injection-site reaction,
Upper respiratory tract infection,
Urinary tract infection,
Back pain,
Fatigue,
Influenza,
Nausea,
Blood immunoglobulin M decreased,
Alopecia,
Arthralgia,
Diarrhoea,
Pain in extremity,
Depression,
Hypertension,
Paraesthesia.

3. Ofatumumab for use according to claim 1 or 2, wherein the history of previous or ongoing conditions other than multiple sclerosis is a history of previous infections.

4. Ofatumumab for use according to claim 3, wherein the previous or ongoing infection was caused by a virus or microorganism selected from the group consisting of
a respiratory syncytial virus (RSV),
an influenza or parainfluenza virus,
a human polyomavirus (BK virus),
an adenovirus,
a rhinovirus,
a corona virus,
a human herpes virus like a herpes simplex virus (HSV),
a varicella zoster virus (VZV),
an Epstein-Barr virus (EBV),
a cytomegalovirus (CMV),
a betapolyomavirus like John Cunningham virus (JCV).
Bordetella pertussis,
Bordetella parapertussis,
Corynebacterium diphtheriae,
E. coli,
Staphylococcus spec. (e.g. Staphylococcus saprophyticus or Staphylococcus aureus),
Chlamydia trachomatis,
Haemophilus influenzae,
Meningococcus spec.,
Klebsiella spec.,
Pseudomonas spec.,
Enterococcus spec.,
Streptococcus spec.
yeast (e.g. Candida albicans),
Pneumocystis spec. (e.g. Pneumocystis murina),
Cryptococcus spec. (e.g. Cryptococcus neoformans),
Aspergillus spec.,
Mycoplasma genitalium.

5. Ofatumumab for use according to any one of the preceding claims, wherein the patient is vaccinated during ofatumumab therapy.

6. Ofatumumab for use according to claim 5, wherein the vaccination is against any one of
a thinoviruses,
a respiratory syncytial virus (RSV),
an influenza or parainfluenza virus,
a human polyomavirus (BK virus),
an adenovirus,
a human herpesvirus like a Herpes simplex virus (HSV),
a varicella zoster virus (VZV),
an Epstein-Barr virus (EBV),
a Cytomegalovirus (CMV),
a betapolyomavirus like John Cunningham virus (JCV),
Bordetella pertussis,
Bordetella parapertussis,
Corynebacterium diphtheriae,
E. coli,
Staphylococcus saprophyticus,
Staphylococcus aureus,
Chlamydia trachomatis,
Klebsiella spec.,
Pseudomonas spec.,
Enterococcus spec.,
Streptococcus spec.,
yeast (e.g. Candida albicans),
Pneumocystis spec. (e.g. Pneumocystis murina),
Cryptococcus spec. (e.g. Cryptococcus neoformans),
Aspergillus spec.,
Mycoplasma genitalium.

7. Ofatumumab for use according to any one of the preceding claims, wherein the patient with a history of previous or ongoing conditions other than multiple sclerosis has a
history of transient ischemic attack (TIA);
history of cerebral infarction without residual deficits;
history of thrombotic stroke without lasting effects;
history of thrombotic stroke without residual deficits;
history of transient ischemic attack;
history of ischemic stroke without residual deficits;
history of nonatherosclerotic stroke without residual deficits;
history of parietal cerebrovascular accident;
history of cerebrovascular accident without residual deficits;
history of embolic stroke without deficits;
history of embolic stroke without lasting effects;
history of embolic transient ischemic attack;
history of haemorrhagic cerebrovascular accident without residual deficits;
history of atherosclerotic cerebrovascular accident without residual deficits;
history of cardioembolic stroke.

8. Ofatumumab for use according to any one of the preceding claims, wherein the patient with a history of previous or ongoing conditions other than multiple sclerosis followed a treatment to cure, alleviate or abolish said condition before commencing ofatumumab therapy.

9. Ofatumumab for use according to claim 8, wherein the treatment comprises administration of glucocorticoids, such as cortisol, and/or nonsteroidal anti-inflammatory drugs (NSAIDs), such as ibuprofen (Motrin, Advil) and naproxen (Naprosyn) or COX-2 inhibitors, such as celecoxib, and/or immune-suppressing drugs.

10. Ofatumumab for use according to any one of the preceding claims, wherein the previous or ongoing condition is an autoimmune disease other than multiple sclerosis.

11. Ofatumumab for use according to claim 10, wherein the autoimmune disease other than multiple sclerosis is selected from
Type 1 diabetes,
Rheumatoid arthritis (RA),
Psoriasis/psoriatic arthritis,
Systemic lupus erythematosus (SLE),
Inflammatory bowel disease,
Addison's disease,
Graves' disease,
Sjögren's syndrome,
Hashimoto's thyroiditis,
Myasthenia gravis,
Autoimmune vasculitis,
Pernicious anemia,
Celiac disease.

12. Ofatumumab for use according to claim 10, wherein the autoimmune disease is rheumatoid arthritis (RA).

13. Ofatumumab for use according to claim 12, wherein the rheumatoid arthritis (RA) is treated with a disease-modifying antirheumatic drug (DMARD) selected from the group of methotrexate, hydroxychloroquine, sulfasalazine, leflunomide, TNF-alpha inhibitors (certolizumab, infliximab and etanercept), abatacept, anakinra, rituximab and tocilizumab.

14. Ofatumumab for use according to claim 10, wherein the autoimmune disease is psoriasis.

15. Ofatumumab for use according to claim 14, wherein the psoriasis is treated with methotrexate, ciclosporin, hydroxycarbamide, fumarates such as dimethyl fumarate, retinoids, anti-TNF therapies such as infliximab, adalimumab, golimumab, and certolizumab pegol, etanercept, ixekizumab, ustekinumab, guselkumab, efalizumab and alefacept.

16. Ofatumumab for use according to any one of the preceding claims, wherein the history of previous or ongoing conditions other than multiple sclerosis is a history of hospitalization.

17. Ofatumumab for use according to any one of the preceding claims, wherein the history of previous or ongoing conditions other than multiple sclerosis is a history of surgery.

18. Ofatumumab for use according to claim 16 or 17, wherein the patient is treated with immunosuppressive agents other than ofatumumab.

19. Ofatumumab for use according to claim 18, wherein the immunosuppressive agents are selected from the group consisting of Glucocorticoids (such as cortisone, prednisone, dexamethasone, and hydrocortisone) Cytostatics (such as methotrexate, anthracyclines, mitomycin C, bleomycin, mithramycin), Antibodies (such as basiliximab (Simulect) and daclizumab (Zenapax)) drugs acting on immunophilins (such as tacrolismus and cyclosporine).

20. Ofatumumab for use according to any one of the preceding claims, wherein the patient is a geriatric patient.

21. Ofatumumab for use according to claim 20, wherein the geriatric patient suffers from age-related macular degeneration (AMD).

22. Ofatumumab for use according to claim 20, wherein the geriatric patient suffers from Alzheimer's disease.

23. Ofatumumab for use according to claim 20, wherein the geriatric patient suffers from atherosclerosis.

24. Ofatumumab for use according to claim 20, wherein the geriatric patient suffers from benign prostatic hyperplasia (BPH).

25. Ofatumumab for use according to any one of claims 1 to 19, wherein ofatumumab is administered to a paediatric patient.

26. Ofatumumab for use according to any one of the preceding claims, wherein the condition other than multiple sclerosis is cancer or a lymphoproliferative disease.

27. Ofatumumab for use according to any one of the preceding claims, wherein ofatumumab is administered when serum neurofilament light chain (NfL) concentration is 4 to 13 pg/mL.

28. Ofatumumab for use according to any one of the preceding claims, wherein ofatumumab is administered at a dose of 10 to 30 mg every 4 weeks, preferably 20 mg every 4 weeks.

29. Ofatumumab for use according to any one of the preceding claims, wherein ofatumumab is administered subcutaneously.

30. Ofatumumab for use according to any one of the preceding claims, wherein ofatumumab is administered with a loading dose.

31. Ofatumumab for use according to claim 30, wherein 20 mg ofatumumab at days 1, 7 and 14 or preferably at week 0, week 1 and week 2 is administered as a loading dose.

32. Ofatumumab for use according to any one of claims 1 to 31, wherein ofatumumab is administered without a loading dose.

33. Ofatumumab for use according to any one of the preceding claims, wherein relapsing multiple sclerosis is selected from relapsing remitting multiple sclerosis (RRMS) and secondary progressive multiple sclerosis (SPMS).

34. Ofatumumab for use according to any one of the preceding claims, wherein a premedication is administered to the patient before the first dose of ofatumumab is administered.

35. Ofatumumab for use according to claim 34, wherein the premedication comprises acetaminophen, antihistamines and/or steroids.

36. Ofatumumab for use according to claim 34 or 35, wherein the premedication is administered 30 to 60 minutes prior to ofatumumab injection.

37. Ofatumumab for use according to any one of claims 1 to 33, wherein no premedication is administered prior to the first dose of ofatumumab.

38. Ofatumumab for use in the treatment or prevention of relapsing multiple sclerosis, wherein a vaccination is performed during ofatumumab therapy.

39. Ofatumumab for use according to claim 38, wherein the vaccination is against any one of
a thinoviruses,
a respiratory syncytial virus (RSV),
an influenza or parainfluenza virus,
a human polyomavirus (BK virus),
an adenovirus,
a human herpesvirus like a Herpes simplex virus (HSV),
a varicella zoster virus (VZV),
an Epstein-Barr virus (EBV),
a Cytomegalovirus (CMV),
a betapolyomavirus like John Cunningham virus (JCV).
Bordetella pertussis,
Bordetella parapertussis,
Corynebacterium diphtheriae,
E. coli,
Staphylococcus saprophyticus,
Staphylococcus aureus,
Chlamydia trachomatis,
Klebsiella spec.,
Pseudomonas spec.,
Enterococcus spec.,
Streptococcus spec.
yeast (e.g. Candida albicans),
Pneumocystis spec. (e.g. Pneumocystis murina),
Cryptococcus spec. (e.g. Cryptococcus neoformans),
Aspergillus spec.
Mycoplasma genitalium
a corona virus, in particular SARS-CoV-2.

40. Ofatumumab for use according to any one of the preceding claims, wherein the patient is neurologically stable within one month prior to the first administration of ofatumumab.

41. Ofatumumab for use according to any one of the preceding claims, wherein the patient has an EDSS score of 1 to 4 prior to the first administration of ofatumumab.

42. Ofatumumab for use according to any one of the preceding claims, wherein ofatumumab is administered at a dose of 10 to 30 mg every 4 weeks, preferably 20 mg every 4 weeks.

43. Ofatumumab for use according to any one of the preceding claims, wherein ofatumumab is administered subcutaneously.

44. Ofatumumab for use according to any one of the preceding claims, wherein ofatumumab is administered with a loading dose.

45. Ofatumumab for use according to claim 44, wherein 20 mg ofatumumab at days 1, 7 and 14 or preferably at week 0, week 1 and week 2 is administered as a loading dose.

46. Ofatumumab for use according to any one of claims 1 to 43, wherein ofatumumab is administered without a loading dose.

47. Ofatumumab for use according to any one of the preceding claims, wherein relapsing multiple sclerosis is selected from relapsing remitting multiple sclerosis (RRMS) and secondary progressive multiple sclerosis (SPMS).

48. Ofatumumab for use according to any one of the preceding claims, wherein a premedication is administered to the patient before the first dose of ofatumumab is administered.

49. Ofatumumab for use according to claim 48, wherein the premedication comprises acetaminophen, antihistamines and/or steroids.

50. Ofatumumab for use according to claim 48 or 49, wherein the premedication is administered 30 to 60 minutes prior to ofatumumab injection.

51. Ofatumumab for use according to any one of claims 38 to 47, wherein no premedication is administered prior to the first dose of ofatumumab.

52. Use of ofatumumab for the manufacture of a medicament for the treatment or prevention of relapsing multiple sclerosis (RMS), wherein ofatumumab is used as defined in any one of the preceding claims.

53. Method a treating or preventing relapsing multiple sclerosis (RMS) by administering ofatumumab as defined in any one of the preceding claims.
